(19) United States
(12) Patent Application Publication
(10) Pub. No.: US 2002/0141966 A1
(43) Pub. Date: Oct. 3, 2002

(54) Compositions for treatment of malignant effusions, and methods of making and using the same

(76) Inventor: Wenbin Dang, Ellicott City, MD (US)

Correspondence Address: FOLEY, HOAG & ELIOT LLP, ONE POST OFFICE SQUARE, BOSTON, MA, 02109, US

(21) Appl. No.: 09/999,257

(22) Filed: Nov. 15, 2001

Related U.S. Application Data
(60) Provisional application No. 60/249,326, filed on Nov. 16, 2000.

(51) Int. Cl. A61K 31/765; A61K 31/337

(52) U.S. Cl. 424/78.37; 514/449

(57) ABSTRACT

The present invention relates to compositions of a biocompatible polymer and an antineoplastic taxane, and methods of using and making the same, for the treatment of malignant effusions. In certain embodiments, the polymer contains phosphorous linkages.

### 1. Related Application Information

This application claims the benefit of priority to Provisional Patent Application 60/249326, filed Nov. 16, 2000, which application is hereby incorporated by reference in its entirety.

### 2. Introduction

a. Overview

Malignant effusions are abnormal collections of protein-rich fluid in the pleural, pericardial or the peritoneal cavities accompanying the spread of cancer to those areas. The physiological effects of malignant effusions parallel the physiological effects of benign effusions. As is the case with a benign effusion, the physiological effects of a malignant effusion may be initially treated by draining the effusion, thereby removing the fluid collection from the particular anatomic cavity. However, because the malignant effusion is caused by the presence of localized malignancy within an anatomic cavity, the effusion tends to recur after it is drained. Thus, the management of malignant effusions tends to be more complicated and less effective than the management of benign effusions. A need exists in the art for more effective treatment modalities for malignant effusions that alleviates symptoms of clinically compromised patients.

Malignant pleural effusions may arise following metastasis from a non-pleural primary tumor, or as a result of local extension from a lung tumor or a pleural-based primary tumor such as a mesothelioma. Malignant pericardial effusions almost always result from metastatic spread from a distant primary site such as breast, ovary or lung. Malignant peritoneal effusions may result from metastases from intraperitoneal or extraperitoneal primary tumors, or from local extension of an intraperitoneal tumor. Metastases that lodge in the liver may also cause malignant peritoneal effusions. When clinically significant, a malignant peritoneal effusion is termed malignant ascites. Malignant pleural, pericardial and peritoneal effusions may require different types of therapeutic interventions because of the differences in the physiologies of the pleural, pericardial and peritoneal spaces. In general, the appearance of a malignant effusion is a hallmark of advanced disease. Treatment of these effusions, therefore, is generally undertaken for palliation of symptoms in a patient already compromised by extensive malignancy.

Certain aspects of fluid transport within body cavities have been well studied. It is known generally that lymphatics carry large molecules and particulate matter away from tissues. Some fluid also flows through lymphatics, acting as a vehicle or solvent for transported substances. Removing fluid in bulk from tissues is usually a function of blood capillaries, however, rather than lymphatics. Fluid removal takes place by a combination of filtration and diffusion. As blood pressure forces fluid from capillaries, the osmotic pressure of plasma proteins sucks the fluid back into the capillaries. Some fluid is retained within the tissues, though, due to the osmotic pressure exerted by large molecules in tissue spaces. Under normal circumstances, only a small amount of fluid remains in the tissues. This fluid, constantly entering the tissues from the bloodstream, may be subsequently reabsorbed by capillaries or may be taken up by the lymphatics along with the large molecules.

Processes of diffusion permit the exchange of molecules across semipermeable membranes like those found in the capillaries. Fluid exchange is also influenced, however, by the presence of large molecules which may pass into tissues through capillary pores or through leaky capillaries. When a condition results in the accumulation of large molecules in the extravascular fluid space, the tissue osmotic pressure rises and counteracts the effect of plasma proteins inside the capillaries, so that filtration is increased and resorption of fluid is decreased. This imbalance only reverses when the tissue tension becomes high enough to counteract the filtering pressure of the capillaries. The normal movement of fluid from the capillaries to the tissues and back brings about the diffusion of oxygen, nutrients and metabolic byproducts throughout these compartments.

A mainstay of treatment of an effusion is drainage. A single drainage procedure for a malignant effusion is rarely successful in durably controlling symptoms, however. Rather, fluid tends to reaccumulate and require re-drainage. Furthermore, the introduction of a drainage device into the fluid collection may simultaneously introduce infectious microorganisms, so that the sterile fluid collection becomes infected, causing a rapid deterioration in the patient's clinical condition that may result in death. Pleural and peritoneal effusions may be successfully treated by implanting a permanent drainage device that may shunt the excessive fluid into the general circulation, but these procedures also have known, potentially life-threatening, complications. Surgical interventions may be undertaken to obliterate the pleural or pericardial space or to introduce chemical agents that have the same effect. These treatments, though, may be ineffective and may compromise, respectively, lung or cardiac function. For recurrent fluid collection, the pericardium may be opened widely to permit its drainage, a surgical or interventional radiological procedure that may be stressful to an already compromised cancer patient. There remains a need in the art, therefore, to provide more effective treatment that avoid the need for interventions on a high frequency and that minimize the risk of complications that accompanies traditional therapies.

b. Malignant Pleural Effusions

Over 50 percent of patients with malignancy will have a pleural effusion during the course of their disease. Lung cancer is the most common primary tumor, followed in frequency by breast and gastrointestinal cancers. Malignant effusions frequently are substantial in volume and in clinical impact. The initial treatment of a malignant pleural effusion is drainage, often by thoracentesis, in hopes that the pleura of the lung will heal to the pleura of the chest wall and that the effusion will not reaccumulate. If the effusion recurs, as it not uncommonly does, the drainage procedure may be repeated, perhaps over a longer period of time by the introduction of a chest tube. In cases of recurrent, symptomatic pleural effusions, indwelling shunts may be placed to drain the pleural cavity into the peritoneum, or surgical interventions may be considered that attach the lung pleura to the chest wall pleura and/or obliterate the pleural space. Shunt placement poses a risk of circulating malignant cells from the pleura into the peritoneum. Shunts, further, require surgery to install and are easily clogged by proteinaceous debris after their implantation. Patients may prevent shunt clogging by compressing a bulb pump, sometimes as often as one hundred times a day. For the majority of end-stage cancer patients with malignant pleural effusions, pleuroperitoneal shunts do not offer meaningful palliation. Pleural decortications are major surgical procedures that remove some of the pleural surface inside the chest cavity so the lung will adhere thereto. A compromised cancer patient may be poorly able to tolerate a procedure of that magnitude. For many patients, chemical pleurodesis offers the most satisfactory relief.

Chemical pleurodesis procedures introduce a pharmacological agent into the pleural space after complete drainage of the fluid so that the fluid will induce inflammatory adhesion between the lung pleura and the thoracic pleura to obliterate the pleural space. While talc is commonly used for chemical pleurodesis, other agents such as tetracycline, doxycycline and bleomycin have also been employed successfully. Chemotherapeutic agents may also be instilled intrapleurally to treat a malignant pleural effusion. A Phase II trial has evaluated the effect of a single intracavitary infusion of paclitaxel to treat malignant effusions associated with non-small cell lung cancer. In that study, using only a single 125 mg/m2 dose of paclitaxel diluted in saline, there was a complete response rate of 28.6%, with a total response rate of 92.9%. Of note, one patient out of fourteen had total tumor mass reduced by more than 50% following the treatment. Another Phase II trial studied the effect of intracavitary infusions of cytosine arabinoside and cisplatin. This study used repeated doses as tolerated of the two agents, reporting an overall response rate of 74%. Pleurodesis treatments must be preceded by complete evacuation of pleural fluid and re-expansion of the lung, carried out by inserting a chest tube and draining the area under water-seal. After the pleurodesis agent is instilled, tube thoracostomy is maintained until pleural fluid output is minimal, usually two to three days. A number of conditions and complications may interfere with successful pleurodesis: loculations, inaccurate chest tube placement, entrapment of the lung by visceral pleural peel, and inadequate fluid removal. There remains in the art, therefore, a need for a more effective regimen for infusing an agent intrapleurally to treat a malignant effusion with a high likelihood of success, with minimal local and systemic side-effects. It is further desirable that such a regimen have a therapeutic effect upon the underlying malignant condition. In addition, it would be advantageous to provide a procedure for treating malignant pleural effusions that is synergistic with systemic modalities of cancer therapy.

c. Malignant Pericardial Effusions

Malignant pericardial effusions involve the accumulation of fluid within the pericardium, a tough membrane surrounding the heart. If fluid accumulates rapidly within the pericardium, it compresses the heart, leading to an acute clinical emergency called cardiac tamponade. More gradual fluid accumulation may still compress the heart, a condition called chronic constrictive pericarditis, wherein venous return and ventricular diastolic filling are restricted, with resultant symptoms of heart failure. Treatment initially requires the removal of fluid from the pericardial sac. If the condition recurs, repeated pericardiocenteses may be undertaken. If the condition persists despite repeated pericardiocentesis, an agent such as tetracycline or bleomycin may be instilled within the pericardial space to induce an inflammatory reaction that obliterates the space, similar to what chemical pleurodesis aims to accomplish with pleural effusions. Alternatively, an aperture may be created in the pericardium using surgical or percutaneous techniques, so that any fluid produced within the pericardial sac will drain out and be unable to compress the heart. While chemotherapeutic agents, notably cisplatin, have been instilled intrapericardially to treat malignant pericardial effusions, their efficacy is limited and repetitive administrations may be required. There remains a need in the art, therefore, for a treatment for malignant pericardial effusions that requires minimal medical intervention and that offers a durable solution to this problem.

d. Malignant Peritoneal Effusions

Malignant peritoneal effusions, like malignant pleural and pericardial effusions, are generally characterized by a production of intracavitary fluid that exceeds the local tissues' ability to remove it. Unlike pleural and pericardial effusions, however, peritoneal effusions collect in a distensible space, the peritoneum. Hence, a considerable amount of fluid may collect before the patient becomes symptomatic. One of the hallmark symptoms of increasing intraperitoneal fluid is an increase in abdominal girth, due to the expansion of the intra-abdominal volume with fluid. As the intra-abdominal fluid volume expands, the patient may experience increasing abdominal discomfort due to the pressure of the fluid on the external abdominal wall and the internal organs. In extreme cases, respiratory function may be affected, because the diaphragm is pushed superiorly by fluid below it that resides in the free peritoneal space.

A clinically significant effusion within the peritoneum is termed ascites. Ascites attributable to a malignant process may be called malignant ascites. The development of ascites is due to changes in both the influx and the efflux of fluid through the peritoneum, resulting in a net accumulation of fluid in the peritoneal cavity. In normal patients, there is a constant movement of fluid into and out of the peritoneum, with a normal transfer of fluid across the peritoneal membrane of approximately 4 to 5 ml per hour. The normal peritoneum is able to reabsorb fluid much faster than fluid is produced, with the result that there is normally only about 50 cc of fluid residing within the peritoneal cavity. Fluidic flux from the peritoneum appears to drain primarily by way of the diaphragmatic lymphatics, with the omental and peritoneal lymphatics and the thoracic duct playing less important roles.

When these substances do not circulate normally, tissues may accumulate toxic waste products, or may lack oxygen or nutrition, all conditions that may lead to tissue damage and necrosis. In the peritoneal cavity, the constant mechanical mixing of ascitic fluid produced by diaphragmatic and intestinal motion facilitates diffusion of these metabolically important substances, so that not only do local cells retain their viability, but also malignant cells tend to remain viable as well.

A number of other, non-malignant conditions may also produce ascites, most notably hepatic cirrhosis. Because malignant ascites is due primarily to these tumor-related mechanisms, treatment modalities appropriate for benign ascites tend to be less effective in treating malignant ascites. Various mechanisms have been identified that explain how the presence of a widely disseminated intra-abdominal tumor contributes to the accumulation of the intraperitoneal ascitic fluid.

In animal models, lymphatic obstruction from disseminated tumor with resultant inhibition of fluid efflux has been seen as a major factor in the development of malignant ascites. However, as fluid in the abdomen increases in volume and intra-abdominal hydrostatic pressure therefore rises, the rate of efflux of fluid from the peritoneal cavity also appears to rise. Hence, reduction of fluid efflux due to lymphatic obstruction cannot alone account for the development of malignant ascites. Other reasons besides blocked lymphatics must account for the net accumulation of fluid in malignant ascites. It has been determined that the composition of peritoneal fluid in malignant ascites is markedly different from that found in normal patients: in normal patients, the protein content of peritoneal fluid is about 20 to 25 percent of plasma levels; by contrast, in malignant ascites the protein content of this abnormal peritoneal fluid is approximately 85 percent of plasma levels. It is postulated that the protein content of the abnormal fluid is due to increased microvascular permeability. A factor (VEGF) that would cause just this phenomenon has been identified in human ascites and in animal models. (Zebrowski BK et al., Ann. Surg. Oncol, 1999 Jun, 6:4, 373-78.) Other similar factors are under investigation. (But L. et al., Scand. J. Clin. Lab. Invest., 1993 Apr. 53:2, 117-24.)

The various forms of benign ascites are generally attributable either to increased hydrostatic pressure or to decreased plasma osmotic pressure or some combination of the two. It has been hypothesized that increased venous pressure may also be a factor in malignant ascites. However, studies have indicated that increased hepatic vein pressure becomes a factor in malignant ascites mainly when significant liver metastases cause some degree of hepatic vein obstruction. Under most circumstances, though, increased hepatic vein pressure is not a factor. In most cases, the pathophysiology underlying malignant ascites is understood to be intraperitoneal fluid production that exceeds peritoneal resorptive capacity.

Malignant ascites is usually associated with a widely disseminated intra-abdominal tumor. Ovarian cancer is the most common primary tumor, accounting for between 30 and 54 percent of cases. Other primary intra-abdominal tumors have also been linked with malignant ascites, including pancreas, colorectal, stomach and uterus cancers. (Sedeghi B et al., Cancer, 2000 Jan., 88:2, 358-63). Extra-abdominal sites of malignancy such as breast, lung and lymphoma, may also be associated with malignant ascites. Depending on the tumor of origin, average duration of survival following diagnosis may be as short as 12 to 20 weeks in the gastrointestinal cancers, or may extend to between 58 and 78 weeks, as seen in lymphatic cancers. Overall duration of survival is poor, however, averaging about 20 weeks from time of diagnosis. In ovarian cancer, the most common tumor producing malignant ascites, mean survival following diagnosis ranges from 30 to 35 weeks. A particularly dismal prognosis exists for those patients with malignant ascites where the primary tumor is unknown. This is the second largest subgroup of malignant ascites patients, accounting for between 13 percent and 22 percent of cases. The survival time for these patients ranges from 1 to 12 weeks in different studies.

Treatments for malignant ascites generally fall into two categories: 1) those treatments intended primarily to reduce the volume of accumulated intraperitoneal fluid; and 2) those treatments intended to decrease intra-abdominal tumor burden, thereby altering the peritoneum's propensity for accumulating fluid. Notably, treatments intended to effect adhesions between the visceral and the parietal peritoneum, analogous to those treatments used for pleural or pericardial effusions, are inappropriate for the treatment of peritoneal effusions because the intra-abdominal organs require free motility during peristalsis. Iatrogenic creation of adhesions within the peritoneal cavity would not only fail to eliminate the production of ascites fluid, but also would introduce the risk of potentially life-threatening intestinal obstruction.

The presence of significant intra-abdominal fluid is responsible for a number of symptoms found in malignant ascites, including abdominal distention, abdominal discomfort or pain, ankle swelling, nausea, vomiting and anorexia. Reducing the amount of intra-abdominal fluid may significantly decrease these symptoms. A primary way to reduce intraperitoneal fluid accumulation involves paracentesis, although other types of treatment are also available. Treatments intended to decrease intra-abdominal tumor burden often involve administering chemotherapeutic agents either systemically or intra-abdominally.

Paracentesis relieves ascites symptoms by removing a clinically significant volume of fluid from the peritoneal cavity. It is generally performed using some type of needle or other drainage device that may withdraw fluid into a collection system. In inserting a needle or cannula into the abdomen, one must be cautious to avoid damaging nearby intra-abdominal structures. Previous surgery, large intra-abdominal tumor masses or other anatomic abnormalities may make paracentesis hazardous, so that ultrasound guidance may be required to identify a safe area to drain. Complications from paracentesis have been reported with some frequency, including secondary peritonitis, pulmonary emboli and hypotension. Deaths have been reported that are directly due to such complications. Even if these major complications are avoided, paracentesis may introduce significant alterations in physiology. The rapid removal of a large volume of ascitic fluid will cause fluid shifts across compartments, leading to possible hyponatremia and to a reduction in circulating blood volume with possible hypotension. Patients undergoing massive paracentesis may require simultaneous intravenous infusion with colloid. Another major problem with significant and repeated paracentesis is protein depletion due to removing the protein-rich ascitic fluid. Decrease in serum protein and albumen are well known to introduce their own set of physiological problems, related in part to decreased plasma osmotic pressure. Finally, by introducing a foreign body into the fluid-filled peritoneum, paracentesis exposes the patient to a small but potentially disastrous risk of peritonitis. While paracentesis has been proven valuable in alleviating symptoms due to tense ascites, its effects are palliative only. Furthermore, the procedure is accompanied by a well-known set of complications and physiologic consequences, some of which may prove fatal and some of which may require hospitalization for appropriate management.

A number of other therapeutic modalities exist that endeavor to reduce the accumulation of fluid within the peritoneum. These treatments have been met with variable and usually limited success. Diuretic therapy, a mainstay of treatment for benign ascites found in conditions like liver cirrhosis, may be useful in malignant ascites if there is a major component of hepatic metastasis with resultant micro-obstruction of hepatic venous circulation. (Sharma S. et al., J. Pain Symptom Manage, 1995 Apr., 10:3, 237-42.) Peritoneal venous shunts, such as the Le Veen shunt, have been used to provide effective palliation of symptoms in malignant ascites. (Gough IR et al., Cancer, 1993 Apr., 71:7, 2377-82.) These shunts are intended to return fluid from the peritoneal cavity back to the systemic circulation. The shunt insertion procedure, however, has a known mortality rate, a high morbidity rate and a substantial cost in both time and money. It is also generally understood that a shunt should be used only for non-gastrointestinal tumors, and then only when other treatments have failed and when the patient is likely to live more than three months after the shunting. (Schumacher D L et al., Ann. Surg. Oncol., 1994 Sep., 1:5, 378-81.) In certain cases, radioactive colloids using phosphorus-32 have been instilled in the peritoneal cavity to treat malignant ascites. This therapy has not been well studied, however, and a variety of obstructive complications have been reported. More contemporary experimental treatments include: 1) the intraperitoneal injection of OK-432 to modulate local biologic response, to destroy tumor cells and to decrease ascites; 2) the use of radioactive labeled monoclonal antibodies (Buckman R et al., Gynecol. Oncol., 1992 Oct., 47:1, 102-09); and 3) the use of metalloproteinase inhibitors to decrease tumor angiogenesis (see U.S. Pat. No. 5,872,152). Studies are still ongoing regarding these treatments. However, a need remains for other effective treatment regiments to provide durable palliation to patients with malignant ascites.

Attempts have been made to treat malignant ascites by treating the underlying malignant condition. Treatments directed at the primary intra-abdominal malignancy may be delivered either systemically or intraperitoneally. In patients with tumors known to be responsive to a particular systemic therapy, such therapy may be initiated. For example, patients with ovarian carcinoma may develop ascites even with stage I or stage II disease. Conventional treatment in these patients includes systemic chemotherapy combined with staging and debulking laparotomy. Even though ascites is present at the time of diagnosis, five-year survivals approaching 50 percent may be achieved in ovarian cancer using this combination of treatments. For more advanced disease, other chemotherapeutic regimens may be useful for symptomatic palliation or for extending survival. (Loggie B W et al., Am. Surg., 1997 Feb., 63:2, 137-43.) Instilling chemotherapeutic agents directly into the peritoneal cavity has the advantage of avoiding problems of systemic toxicity that are encountered with intravenous dosing, although regional complications still exist. (Hagiwara A et al., Anticancer Drug Des., 1993 Dec., 8:6, 463-70; Link K H et al., Cancer Treat. Res., 1996, 81:, 31-40.) Drugs administered into the peritoneal cavity are generally absorbed into the portal circulation and metabolized by the liver, so that high intraperitoneal concentrations of a drug will result in low systemic concentration and therefore low incidence of systemic side effects. However, optimal doses, volumes and schedules of intraperitoneal administration have not been determined for many drugs and for many tumors. The variability of all these factors has made it difficult to construct well-controlled studies to evaluate the use of intraperitoneal chemotherapy as treatment for malignant ascites.

Malignant ascites is a manifestation of advanced malignant disease and is associated with an extremely poor prognosis. (Kehoe C, Oncol. Nurs. Forum, 1991 Apr., 18:3, 523-30.) Despite this poor prognosis, however, patients often require treatment for relief of symptoms. A wide variety of treatments have been advocated for malignant ascites, reflecting their overall inadequacy. The poor overall survival of patients with malignant ascites should not stand in the way of recognizing the importance to the patients of effective palliation and symptom relief during the last several months of their lives. There remains a need in the art for an effective treatment modality for malignant ascites that offers a good rate of success and a low rate of complications. It is furthermore desirable that this treatment modality be synergistic with other anti-neoplastic regimens.

### SUMMARY OF THE INVENTION

It is one object of the present invention to provide palliative treatment for patients with advanced malignancies suffering from malignant effusions. It is a further object to provide these patients with relief of malignant effusions without incurring the risks of severe complications. It is yet another object of the present invention to offer a treatment that is useful for malignant pleural effusions, malignant pericardial effusions and malignant ascites, recognizing the physiological differences among these conditions.

According to the present invention, these objects and other desirable results may be accomplished by instilling into the body cavity afflicted with the malignant effusion a therapeutically effective amount of a composition comprising a biocompatible, optionally biodegradable polymer and an antineoplastic taxane. In certain practices of the present invention, the effusion may be drained. In certain practices of the present invention, the body cavity may be reached or penetrated by an access device, or the composition of the present invention may be delivered into the body cavity by the access device.

In part, the present invention is directed to a polymer system for use in the above-described treatments, such as a biocompatible, optionally biodegradable polymer, comprising paclitaxel, taxotere or an analog thereof, methods for treatment using the subject polymers, and methods of making and using the same.

In certain embodiments, a large percentage of the subject compositions may be an antineoplastic taxane. For example, paclitaxel, taxotere or an analog thereof may comprise 5% to 60% or more of the subject composition, such as at least about 10, 20, 30, 40 or at least about 50% of an antineoplastic taxane.

In certain embodiments, administration of the subject polymers results in sustained release of an encapsulated antineoplastic taxane for a period of time and in an amount that is not possible with other modes of administration of such therapeutic agent.

The subject compositions, and methods of making and using the same, achieve a number of desirable results and features, one or more of which (if any) may be present in any particular embodiment of the present invention: (i) a single dose of a subject composition may achieve the desired therapeutically beneficial response through sustained release of an antineoplastic taxane; (ii) sustained release of an antineoplastic taxane from a biocompatible, biodegradable polymer composition; (iii) novel treatment regimens for treating or preventing malignant effusions using the subject compositions for sustained delivery of an antineoplastic agent; (iv) high levels of loading (by weight), e.g. up to 60% or more, of an antineoplastic taxane in biocompatible, biodegradable polymers; (vi) lyophilization or subjection to an appropriate drying technique such as spray drying of the subject compositions and subsequent rehydration; and (vii) co-encapsulation of therapeutic agents in addition to any antineoplastic taxane in biodegradable polymers.

In one aspect, the subject polymers may be biocompatible, biodegradable or both. In certain embodiments, the subject polymers contain phosphorus linkages, including, for example, phosphate, phosphonate and phosphite. In other embodiments, the monomeric units of the present invention have the structures described in the claims appended below, which are hereby incorporated by reference in their entirety into this Summary. In the subject polymers, an in particular in those embodiments containing a phosphorus linkage, the chemical structure of certain of the monomeric units may be varied to achieve a variety of desirable physical or chemical characteristics, including for example, release profiles or handling characteristics of the resulting polymer composition.

In certain embodiments, other materials may be encapsulated in the subject polymer in addition to paclitaxel, taxotere or an analog thereof to alter the physical and chemical properties of the resulting polymer, including for example, the release profile of the resulting polymer composition for the antineoplastic taxane. Examples of such materials include biocompatible plasticizers, delivery agents, fillers and the like.

The compounds and methods of the present invention may be used in conjunction with antineoplastic agents administered locally or systemically according to dosage schedules known to the art or apparent to skilled artisans using no more than routine experimentation.

The present invention provides a number of methods of making the subject compositions. Examples of such methods include those described in the Exemplification below.

In certain embodiments, the subject compositions are in the form of microspheres. In other embodiments, the subject compositions are in the form of nanospheres. In one aspect, the subject compositions of the present invention may be lyophilized or subjected to another appropriate drying technique such as spray drying and subsequently rehydrated for ready use.

In another aspect, the present invention is directed to methods of using the subject polymer compositions for prophylactic or therapeutic treatment. In certain instances, the subject compositions may be used to prevent a disease or condition. In certain embodiments, use of certain of the subject compositions, which release in a sustained manner an antineoplastic taxane, allow for different treatment regimens than are possible with other modes of administration of such therapeutic agent.

In another aspect, the efficacy of treatment using the subject compositions may be compared to treatment regimens without the sustained release afforded by the subject compositions, e.g., a regiment which an antineoplastic taxane is not encapsulated within a subject polymer. For example, in certain embodiments, treatment with a subject composition is expected to result in fewer hypersensitivity reactions than treatment with an antineoplastic taxane, with or without premedication. Alternatively, treatment with a subject composition may result in an increase in the median survival rate in subjects, and in particular humans.

In another aspect, the subject polymers may be used in the manufacture of a medicament for any number of uses, including for example treating any disease or other treatable condition of a patient. In still other aspects, the present invention is directed to a method for formulating polymers of the present invention in a pharmaceutically acceptable carrier.

In another aspect, the present invention may be spray dried and subsequently rehydrated for ready use or injected as powder using appropriate powder injecting device.

In other embodiments, this invention contemplates a kit including subject compositions, and optionally instructions for their use. Uses for such kits include, for example, therapeutic applications. In certain embodiments, the subject compositions contained in any kit have been lyophilized and require rehydration before use.

The embodiments and practices of the present invention, other embodiments, and their features and characteristics, will be apparent from the description, drawings and claims that follow.

### BRIEF DESCRIPTION OF THE FIGURES

FIG. 1 is a graph showing the change in volume of ascites in control and experimental animals as a function of time following the administration of an antineoplastic taxane composition to the experimental population.

FIG. 2 is a graph illustrating Kaplan-Meier survival curves for experimental animals treated with antineoplastic taxane compositions, where number of survival animals is plotted as a function of time.

FIG. 3 is a graph illustrating Kaplan-Meier survival curves for experimental animals treated with antineoplastic taxane compositions, where percent of surviving animals is plotted as a function of time.

### DETAILED DESCRIPTION OF THE INVENTION

1. Overview

The present invention relates to pharmaceutical compositions for the delivery of paclitaxel, taxotere, or analogs thereof, e.g., for the treatment of malignant effusions, i.e., abnormal collections of fluid in body cavities such as the pleural cavity, the peritoneum or the pericardium associated with or due to localized presence of cancer cells therein. In certain embodiments, biocompatible and optionally biodegradable polymers may be used to allow for sustained release of an encapsulated antineoplastic taxane. The present invention also relates to methods of administering such pharmaceutical compositions, e.g., as part of a treatment regimen, for example, intrapleurally, intrapericardially or intraperitoneally. The present invention also provides for kits whereby said pharmaceutical compositions may be delivered to the body cavity within which a malignant effusion may collect.

In certain aspects, the pharmaceutical compositions, upon contact with body fluids including blood, spinal fluid, pleural fluid, pericardial fluid, ascitic fluid, lymph or the like, release the encapsulated drug over a sustained or extended period (as compared to the release from an isotonic saline solution). Such a system may result in prolonged delivery (over, for example, 2 to 2,000 hours, preferably 4 to 1500 hours) of effective amounts (e.g., 0.0001 mg/kg/hour to 10 mg/kg/hour) of the drug. This dosage form may be administered as is necessary depending on the subject being treated, the severity of the affliction, the judgment of the prescribing physician, and the like.

For treatment of malignant effusions, the pharmaceutical compositions of the present invention are adapted for instillation within the body cavity wherein the malignant effusion may collect. The composition may be formed as a flowable material, insertable into the body cavity. A variety of devices and methods for inserting the composition into the relevant body cavity will be familiar to practitioners of ordinary skill in the art. The composition, alternatively, may be formed as a solid object, insertable into the body cavity. A variety of techniques for inserting a solid object into the relevant body cavity will be likewise familiar to practitioners of ordinary skill in the art.

2. Definitions

For convenience, before further description of the present invention, certain terms employed in the specification, examples, and appended claims are collected here. These definitions should be read in light of the remainder of the disclosure and understood as by a person of skill in the art.

The term “access device” is an art-recognized term and includes any medical device adapted for gaining or maintaining access to a body cavity such as the interpleural space, the pericardial space or the peritoneal space. Such devices are familiar to artisans in the medical and surgical fields. An access device may be a needle, a catheter, a cannula, a trocar, a tubing, a shunt, an endoscope such as a thoracoscope, a laparoscope or any similar system, or any other medical device suitable for entering or remaining positioned within the body cavity.

The terms “antineoplastic” and “antineoplastic agent” are art-recognized, and describe therapeutic agents that prevent the development, maturation, or spread of cells characterized by abnormal malignant growth, e.g., for treating or preventing cancer. “Antineoplastic taxanes”, defined below, and examples of antineoplastic agents. In certain embodiments, an antineoplastic agent used in a composition of the invention is as effective or more effective than paclitaxel or docetaxel, or is at least within an order of magnitude as effective as paclitaxel or docetaxel, e.g., has an ED₅₀ less than ten times the ED₅₀ of paclitaxel or docetaxel.

The terms “biocompatible polymer” and “biocompatibility” when used in relation to polymers are art-recognized. For example, biocompatible polymers include polymers that are neither themselves toxic to the host (e.g., an animal or human), nor degrade (if the polymer degrades) at a rate that produces monomeric or oligomeric subunits or other byproducts at toxic concentrations in the host. In certain embodiments of the present invention, biodegradation generally involves degradation of the polymer in an organism, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in certain embodiments, toxicology of a biodegradable polymer intended for in vivo use, such as implantation or injection into a patient, may be determined after one or more toxicity analyses. It is not necessary that any subject composition have a purity of 100% to be deemed biocompatible; indeed, it is only necessary that the subject compositions be biocompatible as set forth above. Hence, a subject composition may be comprise a polymer comprising 99%, 98%, 97%, 96%, 95%, 90%, 85%, 80%, 75% or even less of biocompatible polymers, e.g., including polymers and other materials and excipients described herein, and still be biocompatible.

To determine whether a polymer or other material is biocompatible, it may be necessary to conduct a toxicity analysis. Such assays are well known in the art. One example of such an assay may be performed with live carcinoma cells, such as GT3TKB tumor cells, in the following manner: the sample is degraded in 1M NaOH at 37° C. until complete degradation is observed. The solution is then neutralized with 1M HCl. About 200 μL of various concentrations of the degraded sample products are placed in 96-well tissue culture plates and seeded with human gastric carcinoma cells (GT3TKB) at 104/well density. The degraded sample products are incubated with the GT3TKB cells for 48 hours. The results of the assay may be plotted as % relative growth vs. concentration of degraded sample in the tissue-culture well. In addition, polymers and formulations of the present invention may also be evaluated by well-known in vivo tests, such as subcutaneous implantations in rats to confirm that they do not cause significant levels of irritation or inflammation at the subcutaneous implantation sites.

The term “biodegradable” is art-recognized, and includes polymers, compositions and formulations, such as those described herein, that are intended to degrade during use. Biodegradable polymers typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to side chain or that connects a side chain to the polymer backbone. For example, a therapeutic agent or other chemical moiety attached as a side chain to the polymer backbone may be released by biodegradation. In certain embodiments, one or the other or both generally types of biodegradation may occur during use of a polymer. As used herein, the term “biodegradation” encompasses both general types of biodegradation.

The degradation rate of a biodegradable polymer often depends in part on a variety of factors, including the chemical identity of the linkage responsible for any degradation, the molecular weight, crystallinity, biostability, and degree of cross-linking of such polymer, the physical characteristics of the implant, shape and size, and the mode and location of administration. For example, the greater the molecular weight, the higher the degree of crystallinity, and/or the greater the biostability, the biodegradation of any biodegradable polymer is usually slower. The term “biodegradable” is intended to cover materials and processes also termed “bioerodible”.

In certain embodiments, if the biodegradable polymer also has a therapeutic agent or other material associated with it, the biodegradation rate of such polymer may be characterized by a release rate of such materials. In such circumstances, the biodegradation rate may depend on not only the chemical identity and physical characteristics of the polymer, but also on the identity of any such material incorporated therein.

The term “body cavity” is an art-recognized term and refers to an anatomically closed space within which body organs are contained, specifically excluding the intrathecal space of the central nervous system. The pleural cavity, the pericardial cavity and the peritoneal cavity are examples of body cavities; these cavities may also be called the interpleural space, the pericardial space and the peritoneal space.

In certain embodiments, polymeric formulations of the present invention biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 6 and 8 having a temperature of between 25 and 37° C. In other embodiments, the polymer degrades in a period of between about one hour and several weeks, depending on the desired application.

The term “drug delivery device” is an art-recognized term and refers to any medical device suitable for the application of a drug or therapeutic agent to a targeted organ or anatomic region. The term includes, without limitation, those formulations of the compositions of the present invention that release the therapeutic agent into the surrounding tissues of an anatomic area. The term further includes those devices that transport or accomplish the instillation of the compositions of the present invention towards the targeted organ or anatomic region, even if the device itself is not formulated to include the composition. As an example, a needle or a catheter through which the composition is inserted into the body cavity is understood to be a drug delivery device. As a further example, a stent or a shunt or a catheter that has the composition included in its substance or coated on its surface is understood to be a drug delivery device.

When used with respect to a therapeutic agent or other material, the term “sustained release” is art-recognized. For example, a subject composition which releases a substance over time may exhibit sustained release characteristics, in contrast to a bolus type administration in which the entire amount of the substance is made biologically available at one time. For example, in particular embodiments, upon contact with body fluids including blood, spinal fluid, pleural fluid, pericardial fluid, peritoneal fluid, lymph or the like, the polymer matrices (formulated as provided herein and otherwise as known to one of skill in the art) may undergo gradual degradation (e.g., through hydrolysis) with concomitant release of any material incorporated therein, e.g., paclitaxel, for a sustained or extended period (as compared to the release from a bolus). This release may result in prolonged delivery of therapeutically effective amounts of any incorporated therapeutic agent. Sustained release will vary in certain embodiments as described in greater detail below.

The term “delivery agent” is an art-recognized term, and includes molecules that facilitate the intracellular delivery of a therapeutic agent or other material. Examples of delivery agents include: sterols (e.g., cholesterol) and lipids (e.g., a cationic lipid, virosome or liposome).

The term “microspheres” is art-recognized, and includes substantially spherical colloidal structures, e.g., formed from biocompatible polymers such as subject compositions, having a size ranging from about one or greater up to about 1000 microns. In general, “microcapsules”, also an art-recognized term, may be distinguished from microspheres, because microcapsules are generally covered by a substance of some type, such as a polymeric formulation. The term “microparticles” is art-recognized, and includes microspheres and microcapsules, as well as structures that may not be readily placed into either of the above two categories, all with dimensions on average of less than 1000 microns. If the structures are less than about one micron in diameter, then the corresponding art-recognized terms “nanosphere,” “nanocapsule,” and “nanoparticle” may be utilized. In certain embodiments, the nanospheres, nancapsules and nanoparticles have an average diameter of about 500, 200, 100, 50 or 10 nm.

A composition comprising microspheres may include particles of a range of particle sizes. In certain embodiments, the particle size distribution may be uniform, e.g., within less than about a 20% standard deviation of the median volume diameter, and in other embodiments, still more uniform or within about 10% of the median volume diameter.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The term “treating” is art recognized and includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “fluid” is art-recognized to refer to a non-solid state of matter in which the atoms or molecules are free to move in relation to each other, as in a gas or liquid. If unconstrained upon application, a fluid material may flow to assume the shape of the space available to it, covering for example, the surfaces of intraperitoneal organs or the parietal surfaces of the peritoneal cavity. Such a material may be termed “flowable.” This term is art-recognized and includes, for example, liquid compositions that are capable of being sprayed into a site; injected with a manually operated syringe fitted with, for example, a 23-gauge needle; or delivered through a catheter. Also included in the term “flowable” are those highly viscous, “gel-like” materials at room temperature that may be delivered to the desired site by pouring, squeezing from a tube, or being injected with any one of the commercially available injection devices that provide injection pressures sufficient to propel highly viscous materials through a delivery system such as a needle or a catheter. When the polymer used is itself flowable, a composition comprising it need not include a biocompatible solvent to allow its dispersion within a body cavity. Rather, the flowable polymer may be delivered into the body cavity using a delivery system that relies upon the native flowability of the material for its application to the desired tissue surfaces. For example, if flowable, a composition comprising polymers according to the present invention it can be injected to form, after injection, a temporary biomechanical barrier to coat or encapsulate internal organs or tissues, or it can be used to produce coatings for solid implantable devices. In certain instances, flowable subject compositions have the ability to assume, over time, the shape of the space containing it at body temperature.

Viscosity is understood herein as it is recognized in the art to be the internal friction of a fluid or the resistance to flow exhibited by a fluid material when subjected to deformation. The degree of viscosity of the polymer may be adjusted by the molecular weight of the polymer; other methods for altering the physical characteristics of a specific polymer will be evident to practitioners of ordinary skill with no more than routine experimentation. The molecular weight of the polymer used in the composition of the invention can vary widely, depending on whether a rigid solid state (higher molecular weights) desirable, or whether a fluid state (lower molecular weights) is desired.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition, or component thereof, from one organ, or portion of the body, to another organ, or portion of the body. Each carrier must be “acceptable” in the sense of being compatible with the other ingredients of the subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” is art-recognized, and includes relatively non-toxic, inorganic and organic acid addition salts of compositions of the present invention, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

A “patient,” “subject,” or “host” to be treated by the subject method may mean either a human or non-human animal, such as primates, mammals, and vertebrates.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “preventing”, when used in relation to a condition, such as a malignant effusion, a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population.

“Radiosensitizer” is defined as a therapeutic agent that, upon administration in a therapeutically effective amount, promotes the treatment of one or more diseases or conditions that are treatable with electromagnetic radiation. In general, radiosensitizers are intended to be used in conjunction with electromagnetic radiation as part of a prophylactic or therapeutic treatment. Appropriate radiosensitizers to use in conjunction with treatment with the subject compositions will be known to those of skill in the art. For example and without limitation, in certain embodiments, antineoplastic agents (including antineoplastic taxanes such as paclitaxel) may act as radiosensitizers.

“Electromagnetic radiation” as used in this specification includes, but is not limited to, radiation having the wavelength of 10-20 to 10 meters. Particular embodiments of electromagnetic radiation of the present invention employ the electromagnetic radiation of: gamma-radiation (10⁻²⁰ to 10⁻¹³ m), x-ray radiation (10⁻¹¹ to 10⁻⁹ m), ultraviolet light (10 nm to 400 nm), visible light (400 nm to 700 nm), infrared radiation (700 nm to 1.0 mm), and microwave radiation (1 mm to 30 cm).

The phrases “systemic administration,” “administered systemically,” “peripheral administration” and “administered peripherally” are art-recognized, and include the administration of a subject composition or other material at a site remote from the disease being treated. Administration of an agent directly into, onto or in the vicinity of a lesion of the disease being treated, even if the agent is subsequently distributed systemically, may be termed “local” or “topical” or “regional” administration, other than directly into the central nervous system, e.g., by subcutaneous administration, such that it enters the patient's system and, thus, is subject to metabolism and other like processes.

The phrase “therapeutically effective amount” is an art-recognized term. In certain embodiments, the term refers to an amount of the therapeutic agent that, when incorporated into a polymer of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a tumor or other target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation.

In certain embodiments, a therapeutically effective amount of a taxane, such as paclitaxel, docetaxel, or an analog thereof, for in vivo use will likely depend on a number of factors, including: the rate of release of the agent from the polymer matrix, which will depend in part on the chemical and physical characteristics of the polymer; the identity of the agent; the mode and method of administration; and any other materials incorporated in the polymer matrix in addition to the taxane.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The terms “incorporated” and “encapsulated” are art-recognized when used in reference to a therapeutic agent, or other material and a polymeric composition, such as a composition of the present invention. In certain embodiments, these terms include incorporating, formulating or otherwise including such agent into a composition which allows for sustained release of such agent in the desired application. The terms may contemplate any manner by which a therapeutic agent or other material is incorporated into a polymer matrix, including for example: attached to a monomer of such polymer (by covalent or other binding interaction) and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to the incorporation of a therapeutic agent or other material and at least one other therapeutic agent or other material in a subject composition.

More specifically, the physical form in which any therapeutic agent or other material is encapsulated in polymers may vary with the particular embodiment. For example, a therapeutic agent or other material may be first encapsulated in a microsphere and then combined with the polymer in such a way that at least a portion of the microsphere structure is maintained. Alternatively, a therapeutic agent or other material may be sufficiently immiscible in the polymer of the invention that it is dispersed as small droplets, rather than being dissolved, in the polymer. Any form of encapsulation or incorporation is contemplated by the present invention, in so much as the sustained release of any encapsulated therapeutic agent or other material determines whether the form of encapsulation is sufficiently acceptable for any particular use.

The term “biocompatible plasticizer” is art-recognized, and includes materials which are soluble or dispersible in the compositions of the present invention, which increase the flexibility of the polymer matrix, and which, in the amounts employed, are biocompatible. Suitable plasticizers are well known in the art and include those disclosed in U.S. Pat. Nos. 2,784,127 and 4,444,933. Specific plasticizers include, by way of example, acetyl tri-n-butyl citrate (c. 20 weight percent or less), acetyl trihexyl citrate (c. 20 weight percent or less), butyl benzyl phthalate, dibutyl phthalate, dioctylphthalate, n-butyryl tri-n-hexyl citrate, diethylene glycol dibenzoate (c. 20 weight percent or less) and the like.

“Small molecule” is an art-recognized term. In certain embodiments, this term refers to a molecule which has a molecular weight of less than about 2000 amu, or less than about 1000 amu, and even less than about 500 amu.

The term “aliphatic” is an art-recognized term and includes linear, branched, and cyclic alkanes, alkenes, or alkynes. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Moreover, the term “alkyl” (or “lower alkyl”) includes both “unsubstituted alkyls” and “substituted alkyls”, the latter of which refers to alkyl moieties having substituents replacing a hydrogen on one or more carbons of the hydrocarbon backbone. Such substituents may include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls may be further substituted with alkyls, alkenyls, alkoxys, alkylthios, aminoalkyls, carbonyl-substituted alkyls, —CF₃, —CN, and the like.

The term “aralkyl” is art-recognized, and includes alkyl groups substituted with an aryl group (e.g., an aromatic or heteroaromatic group).

The terms “alkenyl” and “alkynyl” are art-recognized, and include unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to ten carbons, alternatively from one to about six carbon atoms in its backbone structure. Likewise, “lower alkenyl” and “lower alkynyl” have similar chain lengths.

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “aryl” is art-recognized, and includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and apply to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized, and include 3- to about 10-membered ring structures, such as 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” and “polycyclic group” are art-recognized, and include structures with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art recognized and includes an aromatic or non-aromatic ring in which each atom of the ring is carbon. The flowing art-recognized terms have the following meanings: “nitro” means —NO2; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and include both unsubstituted and substituted amines, e.g., a moiety that may be represented by the general formulas:

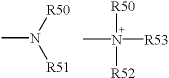

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

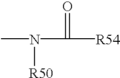

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

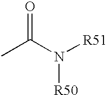

wherein R50 and R51 are as defined above. Certain embodiments of the amide in the present invention will not include imides which may be unstable.

The term “alkylthio” is art recognized and includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art recognized and includes such moieties as may be represented by the general formulas:

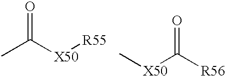

wherein X50 is a bond or represents an oxygen or a sulfur, and R55 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art recognized and include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and includes a moiety that may be represented by the general formula:

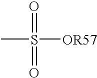

in which R57 is an electron pair, hydrogen, alkyl, cycloalkyl, or aryl.

The term “sulfate” is art recognized and includes a moiety that may be represented by the general formula:

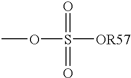

in which R57 is as defined above.

The term “sulfonamido” is art recognized and includes a moiety that may be represented by the general formula:

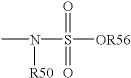

in which R50 and R56 are as defined above.

The term “sulfamoyl” is art-recognized and includes a moiety that may be represented by the general formula:

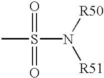

in which R50 and R51 are as defined above.

The term “sulfonyl” is art recognized and includes a moiety that may be represented by the general formula:

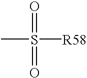

in which R58 is one of the following: hydrogen, alkyl, alkenyl, alkynyl, cycloalkyl, heterocyclyl, aryl or heteroaryl.

The term “sulfoxido” is art recognized and includes a moiety that may be represented by the general formula:

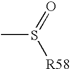

in which R58 is defined above.

The term “phosphoramidite” is art recognized and includes moieties represented by the general formulas:

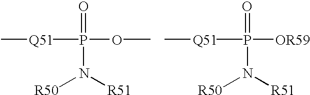

wherein Q51, R50, R51 and R59 are as defined above.

The term “phosphonamidite” is art recognized and includes moieties represented by the general formulas:

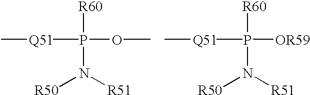

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, etc., when it occurs more than once in any structure, is intended to be independent of its definition elsewhere in the same structure unless otherwise indicated expressly or by the context.

The term “selenoalkyl” is art recognized and includes an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms are art recognized and represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain monomeric subunits of the present invention may exist in particular geometric or stereoisomeric forms. In addition, polymers and other compositions of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, Handbook of Chemistry and Physics, 67th Ed., 1986-87, inside cover. The term “hydrocarbon” is art recognized and includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

The phrase “protecting group” is art recognized and includes temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed. Greene et al., Protective Groups in Organic Synthesis 2^{nd} ed., Wiley, New York, (1991).

The phrase “hydroxyl-protecting group” is art recognized and includes those groups intended to protect a hydroxyl group against undesirable reactions during synthetic procedures and includes, for example, benzyl or other suitable esters or ethers groups known in the art.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (a) constant. This well known constant is described in many references, for instance, March, Advanced Organic Chemistry 251-59, McGraw Hill Book Company, New York, (1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

Contemplated equivalents of the polymers, subunits and other compositions described above include such materials which otherwise correspond thereto, and which have the same general properties thereof (e.g., biocompatible, antineoplastic), wherein one or more simple variations of substituents are made which do not adversely affect the efficacy of such molecule to achieve its intended purpose. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

### 3. EXEMPLARY SUBJECT COMPOSITIONS

a. Taxanes and Other Therapeutic Molecules

A subject composition may comprise paclitaxel, docetaxel or an analog thereof. Paclitaxel and docetaxel share a common framework, and differ primarily in the substituents at two sites on this framework, shown as R1 and R2 in Formula I below:

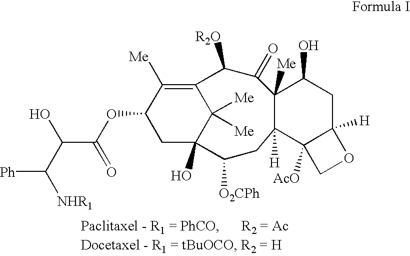

Thus, in one embodiment, a therapeutic composition of the invention comprises a compound of the above formula, wherein R1 is an acyl group or R1—N taken together comprise a carbamyl group (O—C(═O)—N), and R2 is H or an acyl group. In preferred embodiments, R1 comprises between 2 and 12 carbon atoms, preferably between 4 and 9 carbon atoms. In preferred embodiments, R2 is H or an acyl group having between 2 and 8 carbons, preferably between 2 and 4 carbons. In certain embodiments, the therapeutic agent is docetaxel or paclitaxel.

In another embodiment, a therapeutic composition of the present invention includes an antineoplastic compound having a structure of Formula II:

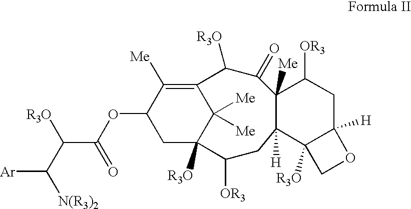

wherein, independently for each occurrence:

Ar represents a substituted or unsubstituted aryl or heteroaryl group; and

R3, each independently, represents H, alkyl, acyl, alkoxycarbonyl, aryloxycarbonyl, aminocarbonyl or sulfonyl.

In certain embodiments, at least one R3 is bound to nitrogen is H or alkyl. In certain embodiments, at least one R3 bound to nitrogen is acyl, alkoxycarbonyl, aryloxycarbonyl, aminocarbonyl, or sulfonyl. In certain embodiments, when R3 is bound to oxygen, R3 is selected from H, alkyl, acyl, aminocarbonyl, alkoxycarbonyl, or aryloxycarbonyl. Preferably, an R3 is selected to be sterically similar to a corresponding substituent on paclitaxel or docetaxel, i.e., contains a number of carbon atoms within four of the number of carbon atoms in a similarly situated substituent of paclitaxel or docetaxel. For example, the benzoate ester of paclitaxel may be exchanged for a tosyl (p-toluenesulfonyl) ester, a cyclohexyl carbamate, or a tetrachlorobenzocyclopentanol carbonate, or a hydroxyl of docetaxel may be exchanged for an ethyl ether, a methylsulfonate ester, or a 2-hydroxyethyl carbamate.

In yet another embodiment, a therapeutic composition of the present invention includes an antineoplastic compound having a structure of Formula III:

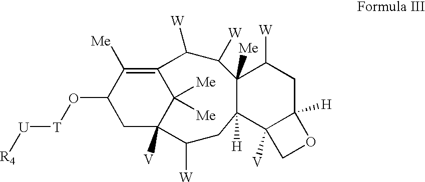

wherein, independently for each occurrence:

V, each independently, represents H, hydroxy, lower alkoxy, or a small ester (e.g., less than 4 carbons);

W, each independently, represents H, hydroxy, carbonyl, amino, alkoxy, sulfhydryl, alkylthio, ester, acylamino, carbamate, sulfonate, carbonate, or sulfoxide;

T represents —C(═O)—, —C(═S)—, —SO₂—, or —SO—;

U is absent or represents NH, S, or O; and

R4 represents a substituted aralkyl.

In certain embodiments, at least one occurrence of W or R4 includes a moiety, such as an oligopeptide or an oligosaccharide, that improves the bioavailability and/or solubility of the taxane. In certain embodiments, the therapeutic compound is formulated as a prodrug, e.g., at least one occurrence of W or R4 includes a moiety capable of being hydrolyzed and cleaved from the molecule under physiological conditions. The hydrolyzable moiety may improve the bioavailability and/or solubility of the taxane. The prodrug form of the therapeutic compound may itself be inactive, provided that after cleavage of the hydrolyzable moiety, the resulting compound is antineoplastic. In certain embodiments, at least one occurrence of W or R4 includes a bond to a polymer, preferably a biocompatible and/or biodegradable polymer. The bond to the polymer may be hydrolyzable under physiologic conditions.

In certain embodiments, a therapeutic composition of the present invention includes an “antineoplastic taxane”, i.e., a compound which has a framework of Formula IV:

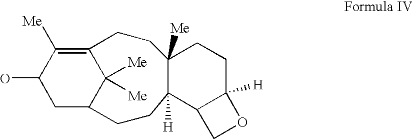

wherein, such framework bears sufficient substituents disposed at unspecified positions, as valence allows, such that the resulting compound has antineoplastic activity. In certain embodiments, such a compound is formed by chemically modifying paclitaxel or 10-deacetylbaccatin III, a naturally occurring compound which has the structure:

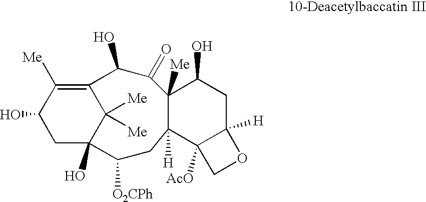

A variety of such antineoplastic derivatives are known in the art, and may be employed in the subject compositions and methods without departing from the spirit or scope of the present invention.

b. Polymers

A variety of polymers may be used in the subject invention. Both non-biodegradable and biodegradable polymers may be used in the subject invention, although biodegradable polymers are preferred. As discussed below, the choice of polymer will depend in part on a variety of physical and chemical characteristics of such polymer and the use to which such polymer may be put.

Representative natural polymers include proteins, such as zein, modified zein, casein, gelatin, gluten, serum albumin, or collagen, and polysaccharides, such as cellulose, dextrans, hyaluronic acid, and polymers of alginic acid.

Representative synthetic polymers include polyphosphazines, poly(vinyl alcohols), polyamides, polycarbonates, polyalkylenes, polyacrylamides, polyanhydrides, poly(phosphoesters), polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyphosphates and polyurethanes.

Synthetically modified natural polymers include alkyl celluloses, hydroxyalkyl celluloses, cellulose ethers, cellulose esters, and nitrocelluloses. Other like polymers of interest include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt.

Representative biodegradable polymers include polylactide, polyglycolide, polycaprolactone, polycarbonate, poly(phosphoesters), polyanhydride, polyorthoesters, and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins.

All of the subject polymers may be provided as copolymers or terpolymers. These polymers may be obtained from chemical suppliers or else synthesized from monomers obtained from these suppliers using standard techniques.

In addition to the listing of polymers above, polymers having phosphorus linkages may be used in the subject invention. Exemplary phosphorus linkages in such polymers include, without limitation, phosphonamidite, phosphoramidite, phosphorodiamidate, phosphomonoester, phosphodiester, phosphotriester, phosphonate, phosphonate ester, phosphorothioate, thiophosphate ester, phosphinate or phosphite. Certain of such polymers may be biodegradable, biocompatible or both.

The structure of certain of the foregoing polymers having phosphorus linkages may be identified as follows. The term “polymer having phosphorous-based linkages” is used herein to refer to polymers in which the following substructure is present at least a multiplicity of times in the backbone of such polymer:

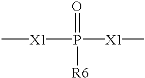

wherein, independently for each occurrence of such substructure:

X1, each independently, represents —O— or —(R5)—;

R5 represents —H, aryl, alkenyl or alkyl; and

R6 is any non-interfering substituent,

wherein such substructure is responsible in part for biodegradability properties (if any) observed for such polymer in vitro or in vivo. In certain embodiments, R6 may represent an alkyl, aralkyl, alkoxy, alkylthio, or alkylamino group.

In certain embodiments, such a biodegradable polymer is non-naturally occurring, i.e., a man-made product with no natural source. In other embodiments, R6 is other than —OH or halogen, e.g., is alkyl, aralkyl, aryl, alkoxy, aralkyoxy or aryloxy. In still other embodiments, the two X1 moieties in such substructure are the same. For general guidance, when reference is made to the “polymer backbone chain” or the like of a polymer, with reference to the above structure, such polymer backbone chain comprises the motif [—X1—P—X1—]. In other polymers, the polymer backbone chain may vary as recognized by one of skill in the art.

By way of example, but not limitation, a number of representative polymers having phosphorus linkages are described in greater detail below. In certain embodiments, a polymer includes one or more monomeric units of Formula V:

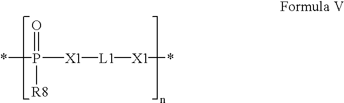

wherein, independently for each occurrence of such unit:

X1, each independently, represents —O— or —N(R7)—;

R7 represents —H, aryl, alkenyl or alkyl;

Li is described below;

R8 represents, for example, —H, alkyl, —O-alkyl, —O-cycloalkyl, aryl, —O-aryl, heterocycle, —O-heterocycle, —N(R9)R10 and other examples presented below;

R9 and R10, each independently, represent a hydrogen, an alkyl, an alkenyl, —(CH2)ₘ—R11, or R9 and R10, taken together with the N atom to which they are attached complete a heterocycle having from 4 to about 8 atoms in the ring structure;

m represents an integer from 0-10, preferably from 0-6; and

R11 represents —H, alkyl, aryl, cycloalkyl, cycloalkenyl, heterocycle or polycycle.

As described in the Summary of the Invention, L1 may be any chemical moiety as long as it does not materially interfere with the polymerization, biocompatibility or biodegradation (or any combination of those three properties) of the polymer, wherein a “material interference” or “non-interfering substituent” is understood to mean: (i) for synthesis of the polymer by polymerization, an inability to prepare the subject polymer by methods known in the art or taught herein; (ii) for biocompatibility, a reduction in the biocompatibility of the subject polymer so as to make such polymer impracticable for in vivo use; and (iii) for biodegradation, a reduction in the biodegradation of the subject polymer so as to make such polymer impracticable for biodegradation.

In certain embodiments, L1 is an organic moiety, such as a divalent branched or straight chain or cyclic aliphatic group or divalent aryl group, with in certain embodiments, from 1 to about 20 carbon atoms. In certain embodiments, L1 represents a moiety between about 2 and 20 atoms selected from carbon, oxygen, sulfur, and nitrogen, wherein at least 60% of the atoms are carbon. In certain embodiments, L1 may be an alkylene group, such as methylene, ethylene, 1,2-dimethylethylene, n-propylene, isopropylene, 2,2-dimethylpropylene, n-pentylene, n-hexylene, n-heptylene; an alkenylene group such as ethenylene, propenylene, 2-(3-propenyl)-dodecylene; and an alkynylene group such as ethynylene, proynylene, 1-(4-butynyl)-3-methyldecylene; and the like. Such unsaturated aliphatic groups may be used to cross-link certain embodiments of the present invention.

Further, L1 may be a cycloaliphatic group, such as cyclopentylene, 2-methylcyclopentylene, cyclohexylene, cyclohexylenedimethylene, cyclohexenylene and the like. L1 may also be a divalent aryl group, such as phenylene, benzylene, naphthalene, phenanthrenylene and the like. Further, L1 may be a divalent heterocyclic group, such as pyrrolylene, furanylene, thiophenylene, alkylyene-pyrrolylene-alkylene, pyridinylene, pyrimidinylene and the like.

Other examples of L1 may include any of the polymers listed above, including the biodegradable polymers listed above, and in particular polylactide, polyglycolide, polycaprolactone, polycarbonate, polyethylene terephthalate, polyanhydride and polyorthoester, and polymers of ethylene glycol, propylene glycol and the like. Embodiments containing such polymers for L1 may impart a variety of desired physical and chemical properties.

The foregoing, as with other moieties described herein, may be substituted with a non-interfering substituent, for example, a hydroxy-, halogen-, or nitrogen-substituted moiety.

R8 represents hydrogen, alkyl, cycloakyl, —O-alkyl, —O-cycloalkyl, aryl, —O-aryl, heterocycle, —O-heterocycle, ester radical, —N(R9)R10, or any other non-interfering subsitutent. Examples of possible alkyl R8 groups include methyl, ethyl, n-propyl, 1-propyl, n-butyl, tert-butyl, -C₈H₁₇ and the like groups; and alkyl substituted with a non-interfering substituent, such as hydroxy, halogen, alkoxy or nitro; corresponding alkoxy groups.

When R8 is aryl or the corresponding aryloxy group, it typically contains from about 5 to about 14 carbon atoms, or about 5 to about 12 carbon atoms, and optionally, may contain one or more rings that are fused to each other. Examples of particularly suitable aromatic groups include phenyl, phenoxy, naphthyl, anthracenyl, phenanthrenyl and the like.

When R8 is heterocyclic or heterocycloxy, it typically contains from about 5 to about 14 ring atoms, alternatively from about 5 to about 12 ring atoms, and one or more heteroatoms. Examples of suitable heterocyclic groups include furan, thiophene, pyrrole, isopyrrole, 3-isopyrrole, pyrazole, 2-isoimidazole, 1,2,3-triazole, 1,2,4-triazole, oxazole, thiazole, isothiazole, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-dioxazole, 1,2,4-dioxazole, 1,3,2-dioxazole, 1,3,4-dioxazole, 1,2,5-oxatriazole, 1,2-pyran, 1,4-pyran, 1,2-pyrone, 1,4-pyrone, 1,2-dioxin, 1,3-dioxin, pyridine, N-alkyl pyridinium, pyridazine, pyrimidine, pyrazine, 1,3,5-triazine, 1,2,4-triazine, 1,2,3-triazine, 1,2-oxazine, 1,3-oxazine, 1,4-oxazine, o-isoxazine, p-isoxazine, 1,2,5-oxathiazine, 1,2,6-oxathiazine, 1,4,2-oxadiazine, 1,3,5-oxadiazine, azepine, oxepin, thiepin, indene, isoindene, benzofuran, isobenzofuran, thionaphthene, isothionaphthene, indole, indolenine, 2-isobenzazole, isoindazole, indoxazine, benzoxazole, anthranil, 1,2-benzopyran, 1,2-benzopyrone, 1,4-benzopyrone, 2,1-benzopyrone, 2,3-benzopyrone, quinoline, isoquinoline, 12,-benzodiazine, 1,3-benzodiazine, naphthyridine, pyrido-[3,4-b]-pyridine, pyrido-[3,2-b]-pyridine, pyrido-[4,3-b]-pyridine, 1,3,2-benzoxazine, 1,4,2-benzoxazine, 2,3,1-benzoxazine, 3,1,4-benzoxazine, 1,2-benzisoxazine, 1,4-benzisoxazine, carbazole, xanthrene, acridine, purine, and the like. In certain embodiments, when R8 is heterocyclic or heterocycloxy, it is selected from the group consisting of furan, pyridine, N-alkylpyridine, 1,2,3- and 1,2,4-triazoles, indene, anthracene and purine rings.

In certain embodiments, R8 is an alkyl group, an alkoxy group, a phenyl group, a phenoxy group, a heterocycloxy group, or an ethoxy group.

In still other embodiments, R8, such as an alkyl, may be conjugated to a bioactive substance to form a pendant drug delivery system.

In certain embodiments, the number of monomeric units in Formula V and other subject formulas that make up the subject polymers ranges over a wide range, e.g., from about 5 to 25,000 or more, but generally from about 100 to 5000, or 10,000. Alternatively, in other embodiments, the number of monomeric units may be about 10, 25, 50, 75, 100, 150, 200, 300 or 400.

In Formula V and other formulas herein, “*” represents other monomeric units of the subject polymer, which may be the same or different from the unit depicted in the formula in question, or a chain terminating group, by which the polymer terminates. Examples of such chain terminating groups include monofunctional alcohols and amines.

In another aspect, the polymeric compositions of the present invention include one or more recurring monomeric units represented in general Formula VI:

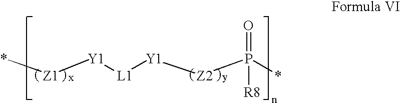

wherein Z1 and Z2, respectively, for each independent occurrence is:

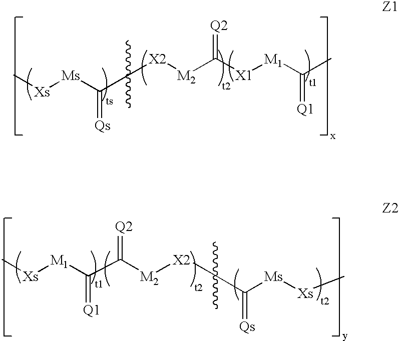

wherein, independently for each occurrence set forth above:

Q1, Q2 . . . Qs, each independently, represent O or N(R1);

X1, X2 . . . Xs, each independently, represent —O— or —N(R1);

the sum of t1, t2 . . . ts is an integer and at least one or more;

Y1 represents —O—, —S— or —N(R7)—;

x and y are each independently integers from 1 to about 1000 or more;

L1 and M1, M2 . . . Ms each independently, represent the moieties discussed below; and

the other moieties are as defined above.

As described in the Summery of the Invention, M1, M2 . . . Ms (collectively, M) in Formula VI are each independently any chemical moiety that does not materially interfere with the polymerization, biocompatibility or biodegradation (or any combination of those three properties) of the subject polymer. For certain embodiments, M in the formula are each independently: (i) a branched or straight chain aliphatic or aryl group having from 1 to about 50 carbon atoms, or (ii) a branched or straight chain, oxa-, thia-, or aza-aliphatic group having from 1 to about 50 carbon atoms, both optionally substituted. In certain embodiments, the number of such carbon atoms does not exceed 20. In other embodiments, M may be any divalent aliphatic moiety having from 1 to about 20 carbon atoms, including therein from 1 to about 7 carbon atoms.

M may include an aromatic or heteroaromatic moiety, optionally with non-interfering substituents. In certain embodiments, none of the atoms (usually but not always C) that form the cyclic ring that gives rise to the aromatic moiety are part of the polymer backbone chain.

Specifically, when M is a branched or straight chain aliphatic group having from 1 to about 20 carbon atoms, it may be, for example, an alkylene group such as methylene, ethylene, 1-methylethylene, 1,2-dimethylethylene, n-propylene, trimethylene, isopropylene, 2,2-dimethylpropylene, n-pentylene, n-hexylene, n-heptylene, n-octylene, n-nonylene, n-decylene, n-undecylene, n-dodecylene, and the like; an alkenylene group such as n-propenylene, 2-vinylpropylene, n-butenylene, 3-thexylbutylene, n-pentenylene, 4-(3-propenyl)hexylene, n-octenylene, 1-(4-butenyl)-3-methyldecylene, 2-(3-propenyl)dodecylene, hexadecenylene and the like; an alkynylene group, such as ethynylene, propynylene, 3-(2-ethynyl)pentylene, n-hexynylene, 2-(2-propynyl)decylene, and the like; or any alkylene, alkenylene or alkynylene group, including those listed above, substituted with a materially non-interfering substituent, for example, a hydroxy, halogen or nitrogen group, such as 2-chloro-n-decylene, 1-hydroxy-3-ethenylbutylene, 2-propyl-6-nitro-10-dodecynylene, and the like. Other M of the present invention include —CH2)₃—, —(CH₂)₅— and —(CH₂)₂OCH₂—.

When M is a branched or straight chain oxaaliphatic group having from 1 to about 20 carbon atoms, it may be, for example, a divalent alkoxylene group, such as ethoxylene, 2-methylethoxylene, propoxylene, butoxylene, pentoxylene, dodecyloxylene, hexadecyloxylene, and the like. When M is a branched or straight chain oxaaliphatic group, it may have the formula —(CH₂)ₐ—O—(CH₂)_{b}— wherein each of a and b, independently, is about 1 to about 7.

When M is a branched or straight chain oxaaliphatic group having from 1 to about 20 carbon atoms, it may also be, for example, a dioxaalkylene group such as dioxymethylene, dioxyethylene, 1,3-dioxypropylene, 2- methoxy-1,3-dioxypropylene, 1,3-dioxy-2-methylpropylene, dioxy-n-pentylene, dioxy-n-octadecylene, methoxylene-methoxylene, ethoxylene-methoxylene, ethoxylene-ethoxylene, ethoxylene-1-propoxylene, butoxylene-n-propoxylene, pentadecyloxylene-methoxylene, and the like. When M is a branched or straight chain, dioxyaliphatic group, it may have the formula —(CH₂)ₐ—O—(CH₂)_{b}—O—(CH₂)_{c}—, wherein each of a, b, and c is independently from 1 to about 7.

When M is a branched or straight chain thiaaliphatic group, the group may be any of the preceding oxaaliphatic groups wherein the oxygen atoms are replaced by sulfur atoms.

When M is a branched or straight chain, aza-aliphatic group having from 1 to about 20 carbon atoms, it may be a divalent group such as —CH₂NH—, —(CH₂)₂N—, —CH₂(C₂H₅)N—, -n-C₄H₉NH—, -t-C₄H₉NH—, —CH₂(C₃H₇)N—, —C₂H₅(C₂H₅)N—, —CH₂(C₈H₁₇)N—, —CH₂NHCH₂—, —(CH₂)₂NCH₂—, —CH₂(C₂H₅)NCH₂CH₂—, -n-C₄H₉NHCH₂—, -t-C₄H₉NHCH₂CH₂—, —CH₂(C₃H₇)N(CH₂)₄—, —C₂H₅(C₂H₅)NCH₂—, —CH₂(C₈H₁₇)NCH₂CH₂—, and the like. When M is a branched or straight chain, amino-aliphatic group, it may have the formula —(CH₂)ₐNR1— or —(CH₂)ₐN(R1)(CH₂)_{b}— where R1 is —H, aryl, alkenyl or alkyl and each of a and b is independently from about 1 to about 7.

x and y of Formula VI each independently represent integers in the range of about 1 to about 1000, e.g., about 1, about 10, about 20, about 50, about 100, about 250, about 500, about 750, about 1000, etc.

For Formula VI, the average molar ratio of (x or y):L1, assuming ts is equal to one, may vary greatly, typically between about 75:1 and about 2:1. In certain embodiments, the average molar ratio of (x or y):L1, when ts is equal to one, is about 10:1 to about 4:1, and preferably about 5:1. The molar ratio of x:y may also vary; typically, such ratio is about 1. Other possible embodiments may have ratios of 0.1, 0.25, 0.5, 0.75, 1.5, 2, 3, 4, 10 and the like.

A number of different polymer structures are contemplated by Formula VI. For example, in certain polymers exemplified by Formula VI, when the sum of t1, t2 . . . ts equals one for each of Z1 and Z2 and Q, M and X for each subunit ts are the same, then Formula VI becomes the following Formula VIa:

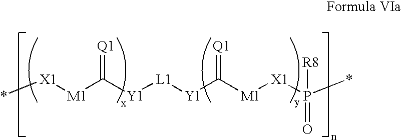

In certain embodiments of Formula VIa (and other subject formulas), x and y may be even integers.

The above Formula VI (and all of the subject formulae and polymers) encompass a variety of different polymer structures, including block copolymers, random copolymers, random terpolymers and segmented block copolymers and terpolymers. Additional structures for Z of subject monomeric units are set forth below, which exemplify in part the variety of structures contemplated by the present invention:

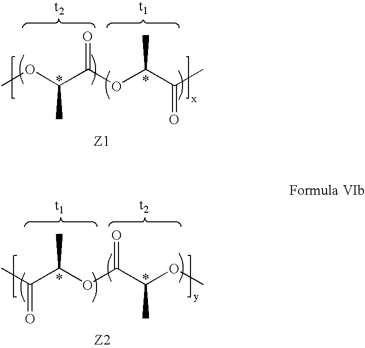

In Formula VIb (and other formulas described below), there may be more ts subunits depicted of the same molecular identity of those depicted in the formulas. For example, in Formula VIb, subunits t₁ and t₂ may be repeated in a sequence, e.g., alternating, in blocks (which may themselves repeat), or in any other pattern or random arrangement. Each subunit may repeat any number of times, and one subunit (e.g., t₁) may occur with substantially the same frequency, more often, or less often than another subunit (e.g., t₂), such that both subunits may be present in approximately the same amount, or in differing amounts, which may differ slightly or be highly disparate, e.g., one subunit is present nearly to the exclusion of the other. In certain embodiments, the chiral centers of each subunit may be the same or different and may be arranged in an orderly fashion or in a random sequence in each of Z1 and Z2.

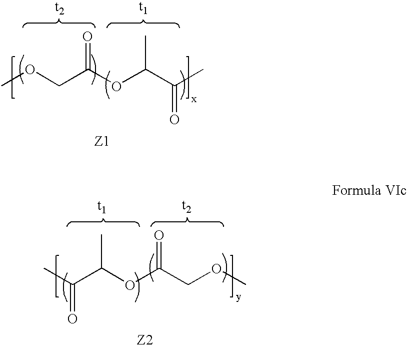

In certain embodiments of Formula VIc, the sum of the number of ts subunits in each of Z1 and Z2 is an even integer. As in other examples of Z1 and Z2, such as described above for Formula VIb, the ts subunits may be distributed randomly or in an ordered arrangement in each of Z1 or Z2.

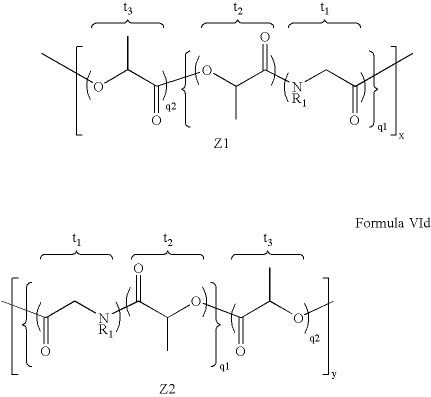

In Formula VId, the subunit q1 is comprised of two ts subunits, which may be repeated and arranged as described above for Formula VIb. In certain embodiments, q2 is an even integer, and in other embodiments, the subunits q1 and q2 may be distributed randomly or in an ordered pattern in each of Z1 and Z2. For example, subunits q₁ and q₂ may be repeated in a sequence, e.g., alternating, in blocks (which may themselves repeat), or in any other pattern or random arrangement. Each subunit may repeat any number of times, and one subunit (e.g., q₁) may occur with substantially the same frequency, more often, or less often than another subunit (e.g., q₂), such that both subunits may be present in approximately the same amount, or in differing amounts, which may differ slightly or be highly disparate, e.g., one subunit is present nearly to the exclusion of the other.

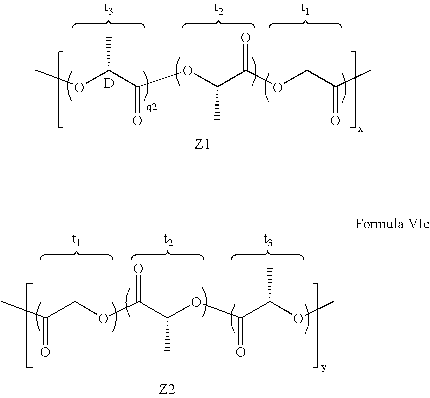

In certain embodiments of Formula VIe, the sum of the ts subunits for each of Z1 and Z2 is an even integer. In other embodiments, the each of the subunits t₁, t₂, and t₃ may be distributed randomly or in an ordered arrangement in each of Z1 and Z2. For example, in Formula VIe, subunits t₁, t₂, and t₃ may be repeated in a sequence, e.g., alternating, in blocks (which may themselves repeat), or in any other pattern or random arrangement. Each subunit may repeat any number of times, and one subunit (e.g., t₁) may occur with substantially the same frequency, more often, or less often than another subunit (e.g., t₃), such that the three subunits may be present in approximately the same amount, or in differing amounts, which may differ slightly or be highly disparate, e.g., two subunits are present nearly to the exclusion of the third.

In certain embodiments of Formula VI, in which Q, M and X for each subunit are the same, Q1 represents O, M represents a lower alkylene group, and X1 represents O or S, preferably O. For example, M may represent —CH(CH₃)— to result in a polymer of Formula VI having a structure represented in Formula VIf:

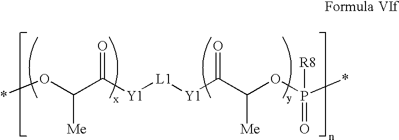

In certain embodiments of Formula VIf, as further described in the Exemplification below, L1 represents a lower alkylene chain, such as ethylene, propylene, etc. In certain embodiments, all Y1's represent O. Incertain embodiments, R8 represents —O-lower alkyl, such as -OEt.

In certain embodiments of polymers depicted by Formula VI, the chirality of each subunit is identical, whereas in other embodiments, the chirality is different. By way of example but not limitation, in Formula VIb above, if the chiral centers of all of the subunits are D-enantiomers or L-enantiomers, then the monomeric unit is effectively equivalent to D-lactic acid or L-lactic acid, respectively, thereby giving rise to a region similar to poly(D-lactic acid) or poly(L-lactic acid), respectively. Conversely, if the two subunits in Formula VIb are comprised of alternating D- and L-enantiomers (e.g., one unit of D-enantiomer, one unit of L-enantiomer, etc.), then the resulting polymeric region is analogous to poly(meso-lactic acid) (i.e., a polymer formed by polymerization of meso-lactide).

Finally, in certain embodiments of the monomeric units set forth in Formula VI, in which the entire polymer may or may not be composed of such units, the following moieties for Y1, L1, R8 Qs, Xs and Ms may be used (with a variety of different x and y being possible):

| Abbreviation | All Y1's | L1 | R8 |
|---|---|---|---|
| L-PL(EG)EOP | O | —CH₂CH₂— | —OCH₂CH₃ |
| L-PL(EG)HOP | O | —CH₂CH₂— | —O(CH₂)₅CH₃ |
| D,L-PL(EG)EOP* | O | —CH₂CH₂— | —OCH₂CH₃ |
| D,L-PL(PG)EOP* | O | —CH₂(CH₃)CH₂— | —OCH₂CH₃ |
| D-PL(PG)EOP | O | —CH₂(CH₃)CH₂— | —OCH₂CH₃ |
| L-PL(PG)EOP | O | —CH₂(CH₃)CH₂— | —OCH₂CH₃ |
| D,L-PL(HD)EOP* | O | 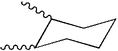 | —OCH₂CH₃ |
| D,L-PL(PG)HOP* | O | —CH₂(CH₃)CH₂— | —O(CH₂)₅CH₃ |
| D,L-PL(PG)EP* | O | —CH₂(CH₃)CH₂— | —CH₂CH₃ |

| Abbreviation | All Qs | All Xs | M1 | M2 |
|---|---|---|---|---|
| L-PL(EG)EOP | O | O | —CH(CH3)-(L) | N/A |
| L-PL(EG)HOP | O | O | —CH(CH3)-(L) | N/A |
| D,L-PL(EG)EOP* | O | O | —CH(CH3)-(L or D) | —CH(CH3)-(D or L) |
| D,L-PL(PG)EOP* | O | O | —CH(CH3)-(L or D) | —CH(CH3)-(D or L) |
| D-PL(PG)EOP | O | O | —CH(CH3)-(D) | N/A |
| L-PL(PG)EOP | O | O | —CH(CH3)-(L) | N/A |
| D,L-PL(HD)EOP* | O | O | —CH(CH3)-(L or D) | —CH(CH3)-(L or D) |
| D,L-PL(PG)HOP* | O | O | —CH(CH3)-(L or D) | —CH(CH3)-(L or D) |
| D,L-PL(PG)EP* | O | O | —CH(CH3)-(L or D) | —CH(CH3)-(L or D) |

*For D,L-PL(EG)EOP, D,L-PL(PG)EOP, D,L-PL(HD)EOP, D,L-PL(PG)HOP, and D,L-PL(PG)EP, if the chiral carbon of M1 has configuration L, then M2 will have configuration D, and vice-versa. The order of the chiral centers in each subunit M1 and M2 for each Z1 and Z2 will be in random order.

In addition to the particular chiral version of the subject polymers described in the above table, polymers in which the chirality of MS varies in each subunit M in the subject polymers are also possible. For instance, referring to D,L-PL(EG)EOP by example, a random order of D and L, in varying amounts, are possible for this polymer. In contrast, the table sets forth one such example in which a D and L chiral M are always adjacent, in equal amounts, but that need not always be the case.

In another embodiment of the present invention, the polymeric compositions of the present invention include one or more recurring monomeric units represented in general Formula VII:

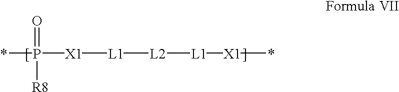

wherein, independently for each occurrence:

L2 is a divalent organic group as described in greater detail below; and

the other moieties are as defined as above.

In Formula VII, L2 may be a divalent, branched or straight chain aliphatic group, a cycloaliphatic group, or a group of the formula:

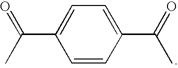

Specific examples of particular divalent, branched or straight chain aliphatic groups include an alkylene group with 1 to 7 carbon atoms, such as 2-methylpropylene or ethylene. Specific examples of cycloaliphatic groups include cycloalkylene groups, such as cyclopentylene, 2-methylcyclopentylene, cyclohexylene and 2-chloro-cyclohexylene; cycloalkenylene groups, such as cyclohexenylene; and cycloalkylene groups having fused or bridged additional ring structures, such as tetralinylene, decalinylene and norpinanylene; or the like.

In certain embodiments of the monomeric units set forth in Formula VII, in which the entire polymer may or may not be composed of such units, the following moieties for X1, L1 and R8 may be used:

| Abbreviation | All X1 | All L1 | L2 | R8 |
|---|---|---|---|---|
| P(trans-CHDM/HOP) | O | —CH2— | 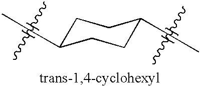 | —O(CH2)5CH3 |
| P(cis- and trans- CHDM/HOP) | O | —CH2— | 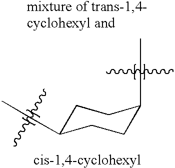 | —O(CH2)5CH3 |
| P(trans-CHDM/BOP) | O | —CH2— | trans-1,4-cyclohexyl | —O(CH2)3CH3 |
| P(trans-CHDM/EOP) | O | —CH2— | trans-1,4-cyclohexyl | —OCH2CH3 |

In another embodiment of the present invention, the polymeric compositions of the present invention include one or more recurring monomeric units represented in general Formula VIII:

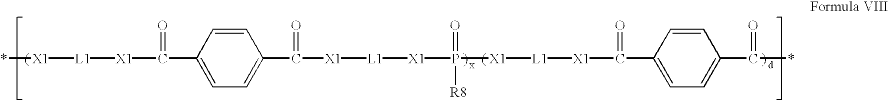

wherein, independently for each occurrence, d is equal to one or more, and optionally two, x is equal to or greater than one, and all of the other moieties are as defined above. In certain embodiments of Formula VIII, each of L1 independently may be an alkylene group, a cycloaliphatic group, a phenylene group or a divalent group of the formula:

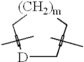

wherein D is O, N or S and m is 0 to 3. Alternatively, L1 is a branched or straight chain alkylyene group having from 1 to 7 carbon atoms, such as a methylene, ethylene, n-propylene, 2-methylpropylene, 2,2′-dimethylpropylene group and the like.

In certain embodiments of the monomeric units set forth in Formula VIII, in which the entire polymer may or may not be composed of such units, the following moieties for X1, L1 and R8 may be used (with a variety of different x possible for each example and with d prefereably equal to two):

| Abbreviation | All X1 | All L1 | R8 |
|---|---|---|---|
| P(BHET-EOP/TC) | O | —CH2CH2— | —OCH2CH3 |
| P(BHDPT-EOP/TC) | O | —CH2CH(CH3)2CH2— | —OCH2CH3 |
| P(BHDPT-HOP/TC) | O | —CH2CH(CH3)2CH2— | —OC6H13 |
| P(BHPT-EOP/TC) | O | —CH2CH2CH2— | —OCH2CH3 |
| P(BHMPT-EOP/TC) | O | CH2CH2(CH3)CH2— | —OCH2CH3 |

In Formula VIII, the aryl groups represented therein may be substituted with a non-interfering substituent, for example, a hydroxy-, halogen-, or nitrogen-substituted moiety.

Other phosphorus containing polymers which may be adapted for use in the subject invention, and methods of making the same, are described in the art, including those described in U.S. Pat. Nos. 5,256,765 and 5,194,581; PCT publications WO 98/44020, WO 98/44021, and WO 98/48859; and U.S. applications Ser. Nos. 09/053,649, 09/053,648 and 09/070,204. For all of the above-identified groups, non-interfering substituents may also be present.

In certain embodiments, the polymers are comprised almost entirely, if not entirely, of the same subunit. Alternatively, in other embodiments, the polymers may be copolymers, in which different subunits and/or other monomeric units are incorporated into the polymer. In certain instances, the polymers are random copolymers, in which the different subunits and/or other monomeric units are distributed randomly throughout the polymer chain. For example, the polymer having units of Formula V may consist of effectively only one type of such subunit, or alternatively two or more types of such subunits. In addition, the polymer may contain monomeric units other than those subunits represented by Formula V.

In other embodiments, the different types of monomeric units, be they one or more subunits depicted by the subject formulas or other monomeric units, are distributed randomly throughout the chain. In part, the term “random” is intended to refer to the situation in which the particular distribution or incorporation of monomeric units in a polymer that has more than one type of monomeric units is not directed or controlled directly by the synthetic protocol, but instead results from features inherent to the polymer system, such as the reactivity, amounts of subunits and other characteristics of the synthetic reaction or other methods of manufacture, processing or treatment.

In certain embodiments, the subject polymers may be cross-linked. For example, substituents of the polymeric chain, may be selected to permit additional inter-chain cross-linking by covalent or electrostatic (including hydrogen-binding or the formation of salt bridges), e.g., by the use of a organic residue appropriately substituted.

The ratio of different subunits in any polymer as described above may vary. For example, in certain embodiments, polymers may be composed almost entirely, if not entirely, of a single monomeric element, such as a subunit depicted in Formula V. Alternatively, in other instances, the polymers are effectively composed of two different subunits, in which the percentage of each subunit may vary from less than 1:99 to more than 99:1, or alternatively 10:90, 15:85, 25:75, 40:60, 50:50, 60:40, 75:25, 85:15, 90:10 or the like. For example, in some instances, a polymer may be composed of two different subunits that may be both represented by the generic Formula V, but which differ in their chemical identity. In certain embodiments, the polymers may have just a few percent, or even less (for example, about 5, 2.5, 1, 0.5, 0.1%) of the subunits having phosphorous-based linkages. In other embodiments, in which three or more different monomeric units are present, the present invention contemplates a range of mixtures like those taught for the two-component systems.

In certain embodiments, the polymeric chains of the subject compositions, e.g., which include repetitive elements shown in any of the subject formulas, have molecular weights ranging from about 2000 or less to about 1,000,000 or more daltons, or alternatively about 10,000, 20,000, 30,000, 40,000, or 50,000 daltons, more particularly at least about 100,000 daltons, and even more specifically at least about 250,000 daltons or even at least 500,000 daltons. Number-average molecular weight (Mn) may also vary widely, but generally fall in the range of about 1,000 to about 200,000 daltons, preferably from about 1,000 to about 100,000 daltons and, even more preferably, from about 1,000 to about 50,000 daltons. Most preferably, Mn varies between about 8,000 and 45,000 daltons. Within a given sample of a subject polymer, a wide range of molecular weights may be present. For example, molecules within the sample may have molecular weights which differ by a factor of 2, 5, 10, 20, 50, 100, or more, or which differ from the average molecular weight by a factor of 2, 5, 10, 20, 50, 100, or more.

One method to determine molecular weight is by gel permeation chromatography (“GPC”), e.g., mixed bed columns, CH₂Cl₂ solvent, light scattering detector, and off-line dn/dc. Other methods are known in the art.

In certain embodiments, the intrinsic viscosities of the polymers generally vary from about 0.01 to about 2.0 dL/g in chloroform at 40° C., alternatively from about 0.01 to about 1.0 dL/g and, occasionally, from about 0.01 to about 0.5 dL/g.

The glass transition temperature (Tg) of the subject polymers may vary widely, and depend on a variety of factors, such as the degree of branching in the polymer components, the relative proportion of phosphorous-containing monomer used to make the polymer, and the like. When the article of the invention is a rigid solid, the Tg is often within the range of from about −10° C. to about 80° C., particularly between about 0 and 50° C. and, even more particularly between about 25° C. to about 35° C. In other embodiments, the Tg is preferably low enough to keep the composition of the invention flowable at body temperature. Then, the glass transition temperature of the polymer used in the invention is usually about 0 to about 37° C., or alternatively from about 0 to about 25° C.

In certain embodiments, substituents of the phosphorus atom, such as R8 in the above formulas, and other components of the subject polymers may permit additional inter-chain cross-linking by covalent or electrostatic interactions (including, for example, hydrogen-binding or the formation of salt bridges) by having a side chain of either of them appropriately substituted as discussed in greater detail below.

In other embodiments, the polymer composition of the invention may be a flexible or flowable material. When the polymer used is itself flowable, the polymer composition of the invention, even when viscous, need not include a biocompatible solvent to be flowable, although trace or residual amounts of biocompatible solvents may still be present.

A flowable polymer composition may be especially suitable for instillation within an irregular body cavity such as the peritoneum. A flowable material is often capable of assuming the shape of the contours of the peritoneum so that it can be applied in certain regions initially and flow therefrom to coat, for example, viscera, the parietal peritoneum or the peritoneal surface of the pelvic organs. A flowable polymer may be particularly adapted for instillation through a needle, catheter or other delivery device such as a laparascope, since its flowable characteristics allow it to reach surfaces that extend beyond the immediate reach of the delivery device. A flowable polymer may also be advantageously employed in body cavities with more regular contours, such as the pleura. Local instillation of a flowable polymer within the pleura would be expected to result in the spread or flowing of said polymer so that it is applied more generally to pleural surfaces. Similarly, local instillation of a flowable polymer within the pericardium may result in its spread into the irregular areas of the pericardial sac, so that more extensive coverage of the affected area is accomplished. Physical properties of polymers may be adjusted to achieve a desirable state of fluidity or flowability by modification of their chemical components and crosslinking, using methods familiar to practitioners of ordinary skill in the art.

A flexible polymer may be used in the fabrication of a solid article. Flexibility involves having the capacity to be repeatedly bent and restored to its original shape. Solid articles made from flexible polymers are adapted for placement in body cavities where they will encounter the motion of adjacent organs or body walls. A flexible solid article can thus be sufficiently deformed by a motile organ structure that it does not cause tissue damage. Flexibility is particularly advantageous where a solid article might be dislodged from its original position and thereby encounter an unanticipated moving structure; flexibility may allow the solid article to bend out of the way of the moving structure instead of injuring it. Physical properties of polymers may be adjusted to attain a desirable degree of flexibility by modification of the chemical components and crosslinking thereof, using methods familiar to practitioners of ordinary skill in the art.

While it is possible that the biodegradable polymer or the biologically active agent may be dissolved in a small quantity of a solvent that is non-toxic to more efficiently produce an amorphous, monolithic distribution or a fine dispersion of the biologically active agent in the flexible or flowable composition, it is an advantage of the invention that, in a preferred embodiment, no solvent is needed to form a flowable composition. Moreover, the use of solvents is preferably avoided because, once a polymer composition containing solvent is placed totally or partially within the body, the solvent dissipates or diffuses away from the polymer and must be processed and eliminated by the body, placing an extra burden on the body's clearance ability at a time when the illness (and/or other treatments for the illness) may have already deleteriously affected it.

However, when a solvent is used to facilitate mixing or to maintain the flowability of the polymer composition of the invention, it should be non-toxic, otherwise biocompatible, and should be used in minimal amounts. Solvents that are toxic clearly should not be used in any material to be placed even partially within a living body. Such a solvent also must not cause substantial tissue irritation or necrosis at the site of administration.

Examples of suitable biocompatible solvents, when used, include N-methyl-2-pyrrolidone, 2-pyrrolidone, ethanol, propylene glycol, acetone, methyl acetate, ethyl acetate, methyl ethyl ketone, dimethylformamide, dimethyl sulfoxide, tetrahydrofuran, caprolactam, dimethyl-sulfoxide, oleic acid, or 1-dodecylazacycloheptan-2-one. Preferred solvents include N-methyl-2-pyrrolidone, 2-pyrrolidone, dimethyl sulfoxide, and acetone because of their solvating ability and their biocompatibility.

The microspheres may be manufactured by incorporating the drug into the polymer matrix by either dissolving or suspending the drug into polymer solution and the mixture will be subsequently dried by techniques familiar to those skill in the arts to form microspheres. These techniques include but not limited to spray drying, coating, various emulsion methods and supercritical fluid processing. The microspheres may be mixed with a pharmaceutically acceptable diluent prior to the administration for injection. They may also be directly applied to the desired site, such as a surgical wound or cavity, by various delivery systems including pouring and spraying. The microspheres may also be mixed with pharmaceutically acceptable ingredients to create ointment or cream for topical applications.

c. Therapeutic Compositions

The antineoplastic agents of the present invention are used in amounts that are therapeutically effective, which varies widely depending largely on the particular antineoplastic agent being used. The amount of antineoplastic agent incorporated into the composition also depends upon the desired release profile, the concentration of the agent required for a biological effect, and the length of time that the biologically active substance has to be released for treatment. In certain embodiments, the biologically active substance may be blended with the polymer matrix of the invention at different loading levels, preferably at room temperature and without the need for an organic solvent. In other embodiments, the compositions of the present invention may be formulated as microspheres.

There is no critical upper limit on the amount of antineoplastic agent incorporated except for that of an acceptable solution or dispersion viscosity to maintain the physical characteristics desired for the composition. The lower limit of the antineoplastic agent incorporated into the delivery system is dependent upon the activity of the drug and the length of time needed for treatment. Thus, the amount of the antineoplastic agent should not be so small that it fails to produce the desired physiological effect, nor so large that the antineoplastic agent is released in an uncontrollable manner. Typically, within these limits, amounts of the antineoplastic agent from about 1% up to about 60% can be incorporated into the present delivery systems. However, lesser amounts may be used to achieve efficacious levels of treatment for antineoplastic agent that are particularly potent.

In addition, the polymer composition of the invention can may comprise blends of the polymer of the invention with other biocompatible polymers or copolymers, so long as the additional polymers or copolymers do not interfere undesirably with the biodegradable or mechanical characteristics of the composition. Blends of the polymer of the invention with such other polymers may offer even greater flexibility in designing the precise release profile desired for targeted drug delivery or the precise rate of biodegradability desired. Examples of such additional biocompatible polymers include other poly(phosphoesters), poly(carbonates), poly(esters), poly(orthoesters), poly(amides), poly(urethanes), poly(imino-carbonates), and poly(anhydrides).

Pharmaceutically acceptable polymeric carriers may also comprise a wide range of additional materials. Without being limited thereto, such materials may include diluents, binders and adhesives, lubricants, disintegrants, colorants, bulking agents, flavorings, sweeteners, and miscellaneous materials such as buffers and adsorbents, in order to prepare a particular medicated composition, with the condition that none of these additional materials will interfere with the intended purpose of the subject composition.

For delivery of an antineoplastic agent or some other biologically active substance, the agent or substance is added to the polymer composition. A variety of methods are known in the art for encapsulating a biologically active substance in a polymer. For example, the agent or substance may be dissolved to form a homogeneous solution of reasonably constant concentration in the polymer composition, or it may be dispersed to form a suspension or dispersion within the polymer composition at a desired level of “loading” (grams of biologically active substance per grams of total composition including the biologically active substance, usually expressed as a percentage).

In part, a biodegradable therapeutic polymer composition of the present invention includes both: (a) paclitaxel, docetaxel, or an analog thereof, such as a compound of Formula I, II, III, or IV, and (b) a biocompatible, and optionally biodegradable, polymer, such as one having the recurring monomeric units shown in one of the foregoing formulas, or any other biocompatible polymer mentioned above or known in the art.

In addition to antineoplastic taxane, the subject compositions may contain a “drug”, “therapeutic agent”, “medicament” or “bioactive substance”, which are biologically, physiologically, or pharmacologically active substances that act locally or systemically in the human or animal body. Various forms of the medicaments or biologically active materials may be used which are capable of being released from the polymer matrix into adjacent tissues or fluids. They may be acidic, basic, or salts. They may be neutral molecules, polar molecules, or molecular complexes capable of hydrogen bonding. They may be in the form of ethers, esters, amides and the like, which are biologically activated when injected into the human or animal body. An antineoplastic taxane is also an example of a “bioactive substance.” Any additional bioactive substance in a subject composition may vary widely with the purpose for the composition. The term bioactive agent includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

Plasticizers and stabilizing agents known in the art may be incorporated in polymers of the present invention. In certain embodiments, additives such as plasticizers and stabilizing agents are selected for their biocompatibility.

A composition of this invention may further contain one or more adjuvant substances, such as fillers, thickening agents or the like. In other embodiments, materials that serve as adjuvants may be associated with the polymer matrix. Such additional materials may affect the characteristics of the polymer matrix that results. For example, fillers, such as bovine serum albumin (BSA) or mouse serum albumin (MSA), may be associated with the polymer matrix. In certain embodiments, the amount of filler may range from about 0.1 to about 50% or more by weight of the polymer matrix, or about 2.5, 5, 10, 25, 40 percent. Incorporation of such fillers may affect the biodegradation of the polymeric material and/or the sustained release rate of any encapsulated substance. Other fillers known to those of skill in the art, such as carbohydrates, sugars, starches, saccharides, celluoses and polysaccharides, including mannitose and sucrose, may be used in certain embodiments in the present invention.

In other embodiments, spheronization enhancers facilitate the production of subject polymeric matrices that are generally spherical in shape. Substances such as zein, microcrystalline cellulose or microcrystalline cellulose co-processed with sodium carboxymethyl cellulose may confer plasticity to the subject compositions as well as implant strength and integrity. In particular embodiments, during spheronization, extrudates that are rigid, but not plastic, result in the formation of dumbbell shaped implants and/or a high proportion of fines, and extrudates that are plastic, but not rigid, tend to agglomerate and form excessively large implants. In such embodiments, a balance between rigidity and plasticity is desirable. The percent of spheronization enhancer in a formulation depends on the other excipient characteristics and is typically in the range of 10-90% (w/w).

Buffers, acids and bases may be incorporated in the subject compositions to adjust their pH. Agents to increase the diffusion distance of agents released from the polymer matrix may also be included.

Disintegrants are substances which, in the presence of liquid, promote the disruption of the subject compositions. Disintegrants are most often used in implants, in which the function of the disintegrant is to counteract or neutralize the effect of any binding materials used in the subject formulation. In general, the mechanism of disintegration involves moisture absorption and swelling by an insoluble material. Examples of disintegrants include croscarmellose sodium and crospovidone which, in certain embodiments, may be incorporated into the polymeric matrices in the range of about 1-20% of total matrix weight. In other cases, soluble fillers such as sugars (mannitol and lactose) can also be added to facilitate disintegration of the subject compositions upon use.

Other materials may be used to advantage to control the desired release rate of a therapeutic agent for a particular treatment protocol. For example, if the sustained release is too slow for a particular application, a pore-forming agent may be added to generate additional pores in the matrix. Any biocompatible water-soluble material may be used as the pore-forming agent. They may be capable of dissolving, diffusing or dispersing out of the formed polymer system whereupon pores and microporous channels are generated in the system. The amount of pore-forming agent (and size of dispersed particles of such pore-forming agent, if appropriate) within the composition should affect the size and number of the pores in the polymer system.

Pore-forming agents include any pharmaceutically acceptable organic or inorganic substance that is substantially miscible in water and body fluids and will dissipate from the forming and formed matrix into aqueous medium or body fluids or water-immiscible substances that rapidly degrade to water-soluble substances. Suitable pore-forming agents include, for example, sugars such as sucrose and dextrose, salts such as sodium chloride and sodium carbonate, and polymers such as hydroxylpropylcellulose, carboxymethylcellulose, polyethylene glycol, and polyvinylpyrrolidone. The size and extent of the pores may be varied over a wide range by changing the molecular weight and percentage of pore-forming agent incorporated into the polymer system.

The charge, lipophilicity or hydrophilicity of any subject polymeric matrix may be modified by attaching in some fashion an appropriate compound to the surface of the matrix. For example, surfactants may be used to enhance wettability of poorly soluble or hydrophobic compositions. Examples of suitable surfactants include dextran, polysorbates and sodium lauryl sulfate. In general, surfactants are used in low concentrations, generally less than about 5%.

Binders are adhesive materials that may be incorporated in polymeric formulations to bind and maintain matrix integrity. Binders may be added as dry powder or as solution. Sugars and natural and synthetic polymers may act as binders. Materials added specifically as binders are generally included in the range of about 0.5%-15% w/w of the matrix formulation. Certain materials, such as microcrystalline cellulose, also used as a spheronization enhancer, also have additional binding properties.

Various coatings may be applied to modify the properties of the matrices. Three exemplary types of coatings are seal, gloss and enteric coatings. Other types of coatings having various dissolution or erosion properties may be used to further modify subject matrices behavior, and such coatings are readily known to one of ordinary skill in the art.

The seal coat may prevent excess moisture uptake by the matrices during the application of aqueous based enteric coatings. The gloss coat generally improves the handling of the finished matrices. Water-soluble materials such as hydroxypropyl cellulose may be used to seal coat and gloss coat implants. The seal coat and gloss coat are generally sprayed onto the matrices until an increase in weight between about 0.5% and about 5%, often about 1% for a seal coat and about 3% for a gloss coat, has been obtained.

Enteric coatings consist of polymers which are insoluble in the low pH (less than 3.0) of the stomach, but are soluble in the elevated pH (greater than 4.0) of the small intestine. Polymers such as EUDRAGIT, RohmTech, Inc., Malden, Mass., and AQUATERIC, FMC Corp., Philadelphia, Pa., may be used and are layered as thin membranes onto the implants from aqueous solution or suspension or by a spray drying method. The enteric coat is generally sprayed to a weight increase of about one to about 30%, preferably about 10 to about 15% and may contain coating adjuvants such as plasticizers, surfactants, separating agents that reduce the tackiness of the implants during coating, and coating permeability adjusters.

The present compositions may additionally contain one or more optional additives such as fibrous reinforcement, colorants, perfumes, rubber modifiers, modifying agents, etc. In practice, each of these optional additives should be compatible with the resulting polymer and its intended use. Examples of suitable fibrous reinforcement include PGA microfibrils, collagen microfibrils, cellulosic microfibrils, and olefinic microfibrils. The amount of each of these optional additives employed in the composition is an amount necessary to achieve the desired effect.

d. Physical Structures of the Subject Compositions

The subject polymers may be formed in a variety of shapes. For example, in certain embodiments, subject polymer matrices may be presented in the form of microparticles or nanoparticles. Such particles may be prepared by a variety of methods known in the art, including for example, solvent evaporation, spray-drying or double emulsion methods.

The shape of microparticles and nanoparticles may be determined by scanning electron microscopy. Spherically shaped nanoparticles are used in certain embodiments for circulation through the bloodstream. If desired, the particles may be fabricated using known techniques into other shapes that are more useful for a specific application.

In addition to intracellular delivery of a therapeutic agent, it also possible that particles of the subject compositions, such as microparticles or nanoparticles, may undergo endocytosis, thereby obtaining access to the cell. The frequency of such an endocytosis process will likely depend on the size of any particle.

In certain embodiments, solid articles useful in defining shape and providing rigidity and structural strength to the polymeric matrices may be used. For example, a polymer may be formed on a mesh or other weave for implantation. A polymer may also be fabricated as a stent or as a shunt, adapted for holding open areas within body tissues or for draining fluid from one body cavity or body lumen into another.

The mechanical properties of the polymer may be important for the processability of making molded or pressed articles for implantation. For example, the glass transition temperature may vary widely but must be sufficiently lower than the temperature of decomposition to accommodate conventional fabrication techniques, such as compression molding, extrusion or injection molding.

e. Biodegradability and Release Characteristics

In certain embodiments, the polymers and blends of the present invention, upon contact with body fluids, undergo gradual degradation. The life of a biodegradable polymer in vivo depends, among other things, upon its molecular weight, crystallinity, biostability, and the degree of crosslinking. In general, the greater the molecular weight, the higher the degree of crystallinity, and the greater the biostability, the slower biodegradation will be.

If a subject polymer matrix is formulated with an antineoplastic taxane or other material, release of such a taxene or other material for a sustained or extended period as compared to the release from an isotonic saline solution generally results. Such release profile may result in prolonged delivery (over, say 1 to about 4,000 hours, or alternatively about 4 to about 1500 hours) of effective amounts (e.g., about 0.00001 mg/kg/hour to about 10 mg/kg/hour) of the antineoplastic taxane or any other material associated with the polymer.

A variety of factors may affect the desired rate of hydrolysis of polymers of the subject invention, the desired softness and flexibility of the resulting solid matrix, rate and extent of bioactive material release. Some of such factors include: the selection of the various substituent groups, such as the phosphate group making up the linkage in the polymer backbone (or analogs thereof), the enantiomeric or diastereomeric purity of the monomeric subunits, homogeneity of subunits found in the polymer, and the length of the polymer. For instance, the present invention contemplates heteropolymers with varying linkages, and/or the inclusion of other monomeric elements in the polymer, in order to control, for example, the rate of biodegradation of the matrix.

To illustrate further, a wide range of degradation rates may be obtained by adjusting the hydrophobicities of the backbones or side chains of the polymers while still maintaining sufficient biodegradability for the use intended for any such polymer. Such a result may be achieved by varying the various functional groups of the polymer. For example, the combination of a hydrophobic backbone and a hydrophilic linkage produces heterogeneous degradation because cleavage is encouraged whereas water penetration is resisted. In another example, it is expected that use of substituent on phosphate in the polymers of the present invention that is lipophilic, hydrophobic or bulky group would slow the rate of degradation. For example, it is expected that conversion of the phosphate side chain to a more lipophilic, more hydrophobic or more sterically bulky group would slow down the rate of biodegradation. Thus, release is usually faster from polymer compositions with a small aliphatic group side chain than with a bulky aromatic side chain.

One protocol generally accepted in the field that may be used to determine the release rate of any therapeutic agent or other material loaded in the polymer matrices of the present invention involves degradation of any such matrix in a 0.1 M PBS solution (pH 7.4) at 37° C., an assay known in the art. For purposes of the present invention, the term “PBS protocol” is used herein to refer to such protocol.

In certain instances, the release rates of different polymer systems of the present invention may be compared by subjecting them to such a protocol. In certain instances, it may be necessary to process polymeric systems in the same fashion to allow direct and relatively accurate comparisons of different systems to be made. For example, the present invention teaches several different means of formulating the polymeric matrices of the present invention. Such comparisons may indicate that any one polymeric system releases incorporated material at a rate from about 2 or less to about 1000 or more times faster than another polymeric system. Alternatively, a comparison may reveal a rate difference of about 3, 5, 7, 10, 25, 50, 100, 250, 500 or 750. Even higher rate differences are contemplated by the present invention and release rate protocols.

In certain embodiments, when formulated in a certain manner, the release rate for polymer systems of the present invention may present as mono- or bi-phasic. Release of any material incorporated into the polymer matrix, which is often provided as a microsphere, may be characterized in certain instances by an initial increased release rate, which may release from about 5 to about 50% or more of any incorporated material, or alternatively about 10, 15, 20, 25, 30 or 40%, followed by a release rate of lesser magnitude.

The release rate of any incorporated material may also be characterized by the amount of such material released per day per mg of polymer matrix. For example, in certain embodiments, the release rate may vary from about 1 ng or less of any incorporated material per day per mg of polymeric system to about 5000 or more ng/day/mg. Alternatively, the release rate may be about 10, 25, 50, 75, 100, 125, 150, 175, 200, 250, 300, 350, 400, 450, 500, 600, 700, 800 or 900 ng/day/mg. In still other embodiments, the release rate of any incorporated material may be 10,000 ng/day/mg or even higher. In certain instances, materials incorporated and characterized by such release rate protocols may include therapeutic agents, fillers, and other substances.

In another aspect, the rate of release of any material from any polymer matrix of the present invention may be presented as the half-life of such material in the such matrix.

In addition to the embodiment involving protocols for in vitro determination of release rates, in vivo protocols, whereby in certain instances release rates for polymeric systems may be determined in vivo, are also contemplated by the present invention. Other assays useful for determining the release of any material from the polymers of the present system are known in the art.

f. Implants and Delivery Systems

In its simplest form, a biodegradable delivery system for an antineoplastic taxane consists of a dispersion of such a therapeutic agent in a polymer matrix. In other embodiments, an article is used for implantation, injection, or otherwise placed totally or partially within the body, the article comprising the subject compositions. It may be particularly important that such an article result in minimal tissue irritation when applied to, implanted in or injected into vascularized tissue. In certain embodiments, a solid, flowable or fluid article comprising the composition of the invention is inserted within a body cavity by implantation, injection, laparoscopy or otherwise being placed within the body cavity of the subject being treated for a malignant effusion accumulating therein.

As a structural medical device, the polymer compositions of the inventions provide a wide variety of physical forms having specific chemical, physical and mechanical properties suitable for insertion into a body cavity, in addition to being a composition that degrades in vivo into non-toxic residues.

Drug delivery systems and articles may be prepared in a variety of ways known in the art. The polymer may be melt processed using conventional extrusion or injection molding techniques, or these products can be prepared by dissolving in an appropriate solvent, followed by formation of the device, and subsequent removal of the solvent by evaporation or extraction, e.g., by spray drying. By these methods, the polymers may be formed into articles of almost any size or shape desired, for example, implantable solid discs or wafers or injectable rods, microspheres, or other microparticles. Typical medical articles also include such as implants as laminates for degradable fabric or coatings to be placed on other implant devices.

In one embodiment, certain polymer compositions of the subject invention may be used to form a soft, drug-delivery “depot” that can be administered as a liquid, for example, by injection, but which remains sufficiently viscous to maintain the drug within the localized area around the injection site. By using a polymer composition in flowable form, even the need to make an incision can be eliminated. In any event, the flexible or flowable delivery “depot” will adjust to the shape of the space it occupies within the body with a minimum of trauma to surrounding tissues.

When the polymer composition of the invention is flexible or flowable, it may be placed anywhere within the body, including into a body cavity. It may be inserted into the body cavity through any of the access devices routinely used in the art to enter such cavities, for example, indwelling or acutely-inserted catheters, needles, chest tubes, peritoneal dialysis catheters and the like. A flowable or fluid polymer may be adapted for mixing with the exudate found within the body cavity with the diagnosis of a malignant effusion. A flowable or fluid polymer may be instilled in body cavities during surgery on organs therein to prevent subsequent malignant effusions when there is a high risk for their development. A polymer according to the present invention may also be incorporated in access devices so that the anti-neoplastic agent is released into the body cavity within which the access device resides, thereby preventing or treating the development of a malignant effusion. The polymer composition of the invention may also be used to produce coatings for other solid implantable devices.

Once a system or implant article is in place, it should remain in at least partial contact with a biological fluid, such as blood, tissue fluid, fluid within body cavities, cerebrospinal fluid or secretions from organ surfaces or mucous membranes, and the like.

g. Exemplary Methods of Making the Subject Polymers

In general, the polymers of the present invention may be prepared by melt polycondensation, solution polymerization or interfacial polycondensation. Techniques necessary to prepare the subject polymers are known in the art, and reference is made in particular to U.S. patent application Ser. No. 09/885,085, filed Jun. 21,2001, which is hereby incorporated by this reference in its entirety.

The most common general reaction in preparing the subject compositions is a dehydrochlorination between a phosphodichloridate and a diol according to the following equation:

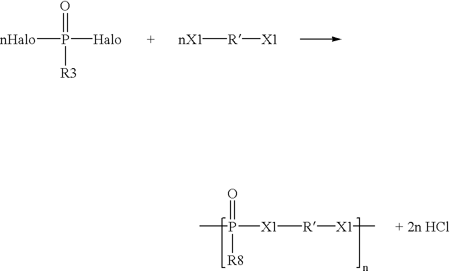

Certain of the subject polymers may be obtained by condensation between appropriately substituted dichlorides and diols.

An advantage of melt polycondensation is that it avoids the use of solvents and large amounts of other additives, thus making purification more straightforward. This method may also provide polymers of reasonably high molecular weight. Somewhat rigorous conditions, however, are often required and may lead to chain acidolysis (or hydrolysis if water is present). Unwanted, thermally induced side reactions, such as cross-linking reactions, may also occur if the polymer backbone is susceptible to hydrogen atom abstraction or oxidation with subsequent macroradical recombination.

To minimize these side reactions, the polymerization may also be carried out in solution. Solution polycondensation requires that both the prepolymer and the phosphorus component be sufficiently soluble in a common solvent. Typically, a chlorinated organic solvent is used, such as chloroform, dichloromethane or dichloroethane. The solution polymerization is generally run in the presence of equimolar amounts of the reactants and, preferably, an excess of an acid acceptor and a catalyst, such as 4-dimethylaminopyridine (DMAP). Useful acid acceptors include tertiary amines as pyridine or triethylamine. The product is then typically isolated from the solution by precipitation in a non-solvent and purified to remove the hydrochloride salt by conventional techniques known to those of ordinary skill in the art, such as by washing with an aqueous acidic solution, e.g., dilute HCl.

Reaction times tend to be longer with solution polymerization than with melt polymerization. However, because overall milder reaction conditions may be used, side reactions are minimized, and more sensitive functional groups may be incorporated into the polymer. The disadvantages of solution polymerization are that removal of solvents can be difficult.

Interfacial polycondensation may be used when high molecular-weight polymers are desired at high reaction rates. By such methods, mild conditions minimize side reactions, and the dependence of high molecular weight on stoichiometric equivalence between diol and dichloridate inherent in solution methods is removed. However, hydrolysis of the acid chloride may occur in the alkaline aqueous phase, and sensitive dichloridates that have some solubility in water are generally subject to hydrolysis rather than polymerization. Phase transfer catalysts, such as crown ethers or tertiary ammonium chloride, may be used to bring the ionized diol to the interface to facilitate the polycondensation reaction. The yield and molecular weight of the resulting polymer after interfacial polycondensation are affected by reaction time, molar ratio of the monomers, volume ratio of the immiscible solvents, the type of acid acceptor, and the type and concentration of the chase transfer catalyst.

Methods for making the present invention may take place at widely varying temperatures, depending upon whether a solvent is used and, if so, which one; the molecular weight desired; the susceptibility of the reactants to form side reactions; and the presence of a catalyst. Usually, the process takes place at a temperature ranging from about 0 to about +235° C. for melt conditions. Somewhat lower temperatures, e.g., for example from about −50 to about 100° C., may be possible with solution polymerization or interfacial polycondensation with the use of either a cationic or anionic catalyst.

The time required for the process may vary widely, depending on the type of reaction being used, the molecular weight desired and, in general, the need to use more or less rigorous conditions for the reaction to proceed to the desired degree of completion. Typically, however, the synthetic process takes place during a time between about 30 minutes and about 7 days.

Although the process may be in bulk, in solution, by interfacial polycondensation, or any other convenient method of polymerization, in many instant embodiments, the process takes place under solution conditions. Particularly useful solvents include methylene chloride, chloroform, tetrahydrofuran, di-methyl formamide, dimethyl sulfoxide or any of a wide variety of inert organic solvents.

In greater detail, polymers of Formula VI may be prepared, at least in part, by reacting a compound having a formula H—Y1—L1—Y1—H, such as 2-aminoethanol, ethylene glycol, ethane dithiol, etc., with a cyclic compound, e.g., having one of the following structures: for example, caprolactone or lactide (lactic acid dimer).

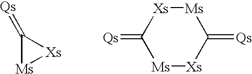

Thus, the cyclic compound may include one or two subunits ts. For cyclic compounds containing two subunits, the two subunits contained therein may be the same or different.

For synthesizing, for example, a compound of Formula VI, wherein x and y are on average about 10, an equivalent of ethylene glycol as H—Y1—L1—Y1—H may be reacted with 20 equivalents of

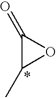

or 10 equivalents of

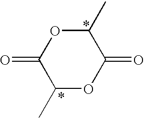

because lactic acid dimer contains two monomer units for each equivalent of the cyclic compound. Variation of the ratio of cyclic compound to ethylene glycol or other bifunctional core will likewise vary the values of x and y, although x and y will be substantially equal for a symmetrical bifunctional core (e.g., ethylene glycol) for subject polymers prepared by this method. For an unsymmetrical bifunctional core (e.g., 2-aminoethanol), the ratio of x:y may vary considerably, as will be understood by one of skill in the art and may be determined without undue experimentation.

Polymers of the present invention may generally be isolated from the reaction mixture by conventional techniques, such as by precipitating out, extraction with an immiscible solvent, evaporation, filtration, crystallization and the like. Typically, the subject polymers are both isolated and purified by quenching a solution of polymer with a non-solvent or a partial solvent, such as diethyl ether or petroleum ether.

In certain embodiments, the subject polymers are soluble in one or more common organic solvents for ease of fabrication and processing. Common organic solvents include such solvents as chloroform, dichloromethane, dichloroethane, 2-butanone, butyl acetate, ethyl butyrate, acetone, ethyl acetate, dimethylacetamide, N-methyl pyrrolidone, dimethylformamide, and dimethylsulfoxide.

### 4. EXEMPLARY METHODS FOR TREATING MALIGNANT EFFUSIONS

a. Overview of Methods

Methods for treating malignant effusions according to the present invention involve gaining access to the body cavity where a malignant effusion is located or may accumulate and instilling therein a composition comprising a biocompatible, optionally biodegradable, polymer and an antineoplastic taxane. According to the present invention, in certain embodiments the polymer composition may be a fluid, a flowable material or a rigid or flexible solid article. Access to the body cavity is gained by techniques familiar to practitioners in the medical arts. Optionally, when access to the body cavity is gained, the malignant effusion fluid may be drained before the polymer is instilled. Alternatively, the polymeric composition may be adapted for mixing with or dissolving in the malignant effusion so that the effusion fluid carries the polymeric composition throughout the extent of the body cavity. In other embodiments, the compositions of the present invention are instilled into the body cavity to prevent or to minimize the accumulation of a malignant effusion in a patient who is at increased risk for developing such an effusion. Optionally, the polymeric composition of the present composition may be removed at a preselected time interval after it is instilled, although certain compositions according to the present invention are formulated to reside within the body cavity for prolonged periods of time or permanently, in certain cases degrading over time or being resorbed by, digested by or metabolized by the local body tissues. Repeated instillations of the subject polymeric compositions may be undertaken, but certain compositions are formulated for sustained or extended release of the therapeutically effective amount of antineoplastic taxane, so that a single applied dose may be sufficient to treat the malignant effusion adequately. Combination therapies for malignant effusions in advanced cancer patients also fall within the scope of the present invention where one component of the combination therapy involves the instillation of the compositions of the present invention as claimed and as described herein. As an example, combined treatment regimens may involve the instillation of an antineoplastic taxane within a body cavity accompanied by another type of treatment, such as systemic chemotherapy administration or locoregional radiation therapy, thermotherapy or other therapeutic application of electromagnetic energy. Other therapeutic combinations, all falling similarly within the scope of the present invention, will be apparent to practitioners of ordinary skill in the art using no more than routine experimentation.

Certain exemplary treatment methods for various body cavities are described below. It is understood, however, that these descriptions are intended as illustrative only, not intended to be limiting in any way, and that other modifications and variations of these illustrative embodiments may be contemplated without departing from the scope of the present invention.

b. Malignant Pleural Effusions

A pleural effusion is an accumulation of fluid in the pleural space. The pleural space is normally only a potential space lying between the visceral pleura surrounding the lung and the parietal pleura of the chest wall. The two pleural surfaces are normally in close apposition despite the elastic recoil of the lung, because of the rapid continuous absorption of fluid from the pleural space. Normally, the balance of hydrostatic and colloid osmotic forces favors movement of fluid out of the pleural space. Between five and ten liters of protein free fluid passes through the pleural space each 24 hours. Lymphatics drain proteins and other substances from the pleural space and thereby remove these sources of colloid osmotic pressure. Alterations in systemic hydrostatic forces or in colloid osmotic pressure can lead to a collection of fluid within the pleural space.

Malignant pleural effusions are understood to result from interference with venous and lymphatic drainage caused by tumor cells. Normal respiratory mechanics may be impaired proportionately to the amount of fluid that collects within the pleural space and to the pressure it exerts. Furthermore, any chronic accumulation of pleural fluid may be associated with other pathophysiological processes that produced thickening and adherence of pleural surfaces, ultimately resulting in adhesions that can cause modulations of fluid or permanent compression of lung parenchyma. A pleural effusion may further impact respiratory mechanics by restricting the normal expansion of the lung on the affected side during inspiration and by interfering with the affected lung's ability to expel its contents during expiration. Malignant effusions may be bilateral, causing further respiratory compromise.

Treatment of a malignant pleural effusion traditionally involves drainage, with pleurodesis techniques reserved for those cases where tube thoracostomy drainage fails to durably resolve the problem. Reaccumulation of fluid is not uncommon in these patients because the cause of the effusion, the presence of tumor cells within the pleural space and on the mesothelial surfaces, remains unaffected by tube thoracostomy drainage. Administration of an antineoplastic taxane may attack the primary problem of intrapleural malignant dissemination so that a drainage procedure will have long-term efficacy in re-expanding the lung.

In one method according to the present invention, the pleural effusion may first be drained either acutely via a thoracentesis or over a longer period of time by tube thoracostomy. A composition comprising a biocompatible (and optionally biodegradable) polymer and an antineoplastic taxane in a therapeutically effective amount may then be instilled into the drained pleural cavity. Access devices permitting instillation of the composition into the pleural space may include a needle, a catheter, a chest tube or a thoracoscopic instrument. In another practice of the instant method, a composition according to the present invention may be instilled in the pleural cavity at surgery to prevent the development of a malignant effusion in high-risk cases such as mesothelioma. In certain embodiments, the polymers available for intra-operative (including thoracoscopic) instillation may be solid articles including meshes and fabrics that can be placed in apposition to the mesothelial surfaces within the pleural cavity. In other embodiments, fluids, sprays, aerosols or gels may be applied, either during open surgery or during an access procedure using an access device. As an alternate delivery system, the access device may itself bear a polymeric composition of the present invention that is then released into the pleural space. A chest tube, for example, may be formulated from a composition of the present invention, or may be coated with such a composition, in a polymeric form such that the antineoplastic taxane, either alone or in combination with a biocompatible, optionally biodegradable polymer, is released into the pleural space. In one embodiment, the chest tube placed to drain the malignant pleural effusion may deliver the entire therapeutically effective amount of the antineoplastic taxane into the pleural space during the several day period when the chest tube is in position. In another embodiment, the chest tube may, during its period of implantation, release into the pleural space from its surface or from its substance a composition comprising a polymer and an antineoplastic taxane according to the present invention; this composition then resides in the pleural space even after chest tube removal and continues to release the antineoplastic taxane as an extended-release substance.

c. Malignant Pericardial Effusions

A pericardial effusion is a collection of fluid within the pericardium. The pericardium, a tough collagenous sac surrounding the heart, is able to expand only gradually in response to a progressive accumulation of fluid therein. Hence, as fluid collects in the pericardium, it can compress the great veins returning blood to the heart, thereby lessening cardiac output and causing backup of venous return into the pulmonary and systemic systems. Acute compression of these structures results in a life-threatening condition called cardiac tamponade. More chronic collection of fluid in the pericardium may nonetheless impair venous return and cardiac output, resulting in symptoms of heart failure.

Malignant pericardial effusions may result from the blockage of the lymphatic and venous outflow from the pericardium that enables the fluid that normally collects in the pericardium to be drained. A symptomatic pericardial effusion may be treated initially by pericardiocentesis. If fluid reaccumulates, a common occurrence with a malignant etiology, more definitive management may be needed. Two general types of treatment are available: adhesion procedures to obliterate the pericardial space, and drainage procedures that allow the efflux of fluid that collects in the pericardium.

In one practice of the present invention, the pericardial effusion may be drained either by needle aspiration (pericardiocentesis) or by a drainage procedure that diverts excess fluid out of the pericardium via a window or that diverts excess fluid from the pericardium to an other body cavity or body lumen. Following drainage, a composition comprising a biocompatible and/or biodegradable polymer and an antineoplastic taxane in a therapeutically effective amount may then be instilled into the drained pericardial space. The composition may be provided as a gel, a fluid, an aerosol or any other physical material that would be suitable for delivery into the pericardial space through an access device or using an access method. In one embodiment, a material deliverable through a catheter or through a needle would be selected. Other physical materials would be chosen if different delivery methods were to be used. For example, a composition formed as a mesh or as a polymeric sheet could be positioned within the pericardial space during the performance of an endoscopic or open pericardial window procedure. In one practice of the present method, a composition according to the disclosed invention may be instilled in the pericardium without draining all the fluid from the space, anticipating that the composition will be readily distributed to all areas within the pericardium by the residual intrapericardial fluid. Under this scenario, a composition could be provided that was soluble in pericardial fluid or that was of sufficient size and shape to be dispersed in pericardial fluid and borne thereby to the anatomic recesses of the pericardial space. In certain embodiments, the compositions available for instillation within the pericardium may be formed as solid articles, such as discs, tubes, coils, rods or wands, that can be implanted using a suitable delivery device. In certain embodiments, the compositions of the present invention may be combined with other medical devices inserted transpericardially for the treatment of cardiac conditions such as arrhythmias that accompany a malignant state. In this situation, an antineoplastic taxane could be incorporated into the manufacture of a pacing system, for example, or some type of implantable anti-arrhythmic device or cardiac assist device; the method of incorporating the antineoplastic taxane into the solid article would then depend upon the particular method of manufacture being used, a determination well within the competence of practitioners of ordinary skill in the art. It is anticipated that the methods of the present invention would be of primary use for the treatment of a malignant pericardial effusion. However, methods for prophylaxis of patients at high risk for developing malignant pericardial effusions fall equally within the scope of the present invention.

d. Malignant Peritoneal Effusions

A peritoneal effusion is a collection of fluid within the peritoneal space. When the magnitude of a peritoneal effusion becomes clinically significant, the fluid collection is called ascites. Massive volumes of fluid can accumulate with malignant ascites, resulting in marked symptomatology. Removing fluid from the peritoneal space, resulting in a decrease in symptoms, is a mainstay of treatment. Most treatment modalities in common use are aimed at removing or diverting the intraperitoneal fluid. Once removed, there is a high likelihood that the fluid will reaccumulate, requiring further removal procedures or requiring a chronic means of ongoing fluid drainage. All these interventions introduce potentially life-threatening complications, including infections, physiological fluid shifts, protein depletion and damage to intra-abdominal organs.

The intra-peritoneal space is characterized by an enormous surface area with a number of anatomic irregularities within which fluid can collect. Furthermore, any part of the peritoneal surface area may be responsible for the production of the ascites fluid. Thus, it may be advantageous for the methods of the present invention to deliver a composition such as an antineoplastic taxane through a delivery system that spreads the composition widely. As an example, the composition may be prepared as an aerosol or a spray to be applied to the surfaces of the visceral and parietal peritoneum. As another example, the composition may be prepared as a liquid to be instilled within the peritoneal cavity. The composition may be formulated as a solid or a gel that is soluble in or miscible with the ascitic fluid, so that the composition may be introduced into a pre-existing volume of ascitic fluid within the peritoneal cavity. In other embodiments, the composition may be formulated as a gel that can be applied to intraperitoneal organs, to be distributed throughout the peritoneal cavity by peristalsis. Alternatively, the composition may be applied to, incorporated into or formulated as part of a fabric or a mesh suitable for residing within the free peritoneal cavity.

A number of methods for applying the composition of the present invention are available, familiar to practitioners of ordinary skill in these arts. The composition, formulated as a fluid or as a flowable material, may be instilled through a catheter or through a needle that is used to aspirate or to drain ascitic fluid. The composition may be applied using laparoscopic instrumentation, whether as a liquid, a gel, a spray or an aerosol. Instruments currently available for laparoscopy or variants thereof specifically adapted for applying the compositions of the present invention may be useful according to the inventive methods. Articles formulated using the compositions of the present invention or articles coated with or bearing the compositions of the present invention may be inserted within the peritoneal cavity in order to release the composition in a sustained or extended release manner. Such articles may have as their primary purpose the release of the inventive compositions, or may have other structural, functional or therapeutic purposes apart from delivery of the inventive compositions. As an example of the latter type of article, a catheter or a stent may be formulated using the compositions of the present invention; such an article may, in addition to releasing the composition, serve additional purposes such as draining fluid or holding open a body lumen. As another example, a permanent shunt for draining ascitic fluid, such as a LeVeen or Denver shunt, may incorporate, be formed from or be coated with a composition according to the present invention. Other examples will be readily discernible by skilled artisans in these fields.

Instillation of compositions according to these inventive methods may accompany procedures for draining ascitic fluid or other surgical procedures. Furthermore, these methods are consistent with prophylactic application, in those cases where the risk of developing malignant ascites is high. For example, in an exploratory surgical procedure where extensive carcinomatosis is apparent, the clinician might deem it advisable to apply the compositions of the present invention within the peritoneal cavity using any of the delivery systems that would be familiar in the art. A liquid, gel, spray, aerosol or formed article could be used under these circumstances to deploy the inventive compositions for the prevention or the minimization of malignant ascites in the future. Such a delivery system could be fabricated and composed to carry out other desirable medical functions without exceeding the scope of the present invention: for example, an antineoplastic taxane composition according to the present invention could be combined with other substances such as anti-adhesion substances, hemostatic substances, immunogenic substances, or any other drug, therapeutic agent or bioactive substance without limitation. Materials bearing the inventive compositions may also be adapted for activation using electromagnetic radiation, including heat energy, light energy and therapeutic radiation delivered from internal or external sources.

### 5. REFERENCES

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Patents and Patent Applications

U.S. Pat. Nos. 4,638,045, 5,219,564, 5,099,060, 6,040,330, 6,017,935, 6,002,023, 5,990,325, 5,981,564, 5,977,164, 5,977,163, 5,972,992, 5,922,754, 5,919,815, 5,908,835, 5,912,263, 5,902,822, 5,877,205, 5,854,278, 5,840,929, 5,821,363, 5,817,840, 5,808,888, 5,795,909, 5,780,653, 5,773,464, 5,773,461, 5,767,297, 5,767,296, 5,760,072, 5,756,776, 5,750,691, 5,739,359, 5,728,687, 5,719,177, 5,693,666, 5,688,977, 5,686,623, 5,670,536, 5,614,645, 5,608,087, 5,597,931, 5,908,835, 6,005,120, 5,424,073, and 5,547,981.

Publications and Other References

Ertel et al., (1995) J. Biomedical Materials Res. 29:1337-1348

Choueka et al., (1996) J. Biomed. Materials Res., 31:35-41)

Langer et al., (1983) Rev. Macro. Chem. Phys. C23(1):61

Leong et al. (1986) Biomaterials, 7:364

Sato et al., (1996) Bio. Pharm. Bull. 19:1596-601

### 6. EXEMPLIFICATION

The invention having been generally described, it will be more readily understood by reference to the following examples which are included for purposes of illustration of certain aspects and embodiments of the present invention and are not intended to limit the invention in any way.

### EXAMPLE 1

### First Synthesis of D,L-PL(PG)EOP

All glassware was dried for a minimum of 2 hours at 105° C. and allowed to cool in a desiccator or cooled under a stream of argon gas. A 28.5 g portion of D,L-lactide and 1.5 g of 1,2-propanediol (PG), obtained from Aldrich, Catalog No. 39,803, 99.5+%, in a molar ratio of 10:1, were weighed into a 250 mL 3-neck round-bottom flask. The flask was equipped with a gas joint and a stirrer bearing/shaft/paddle assembly. The mixture was evacuated and pressurized with argon five times to remove residual air and moisture. The reaction apparatus was immersed in a preheated oil bath at 135° C., connected to an argon source with an oil bubbler, and stirred at a moderate speed until all of the solid monomer had melted. At this time, a volume of stock stannous octoate solution (about 130 mg/ml in toluene of chloroform) equivalent to 3.6 mg tin (120 ppm stannous octoate or equivalent to 35 ppm tin based upon weight of the prepolymer) was added to the melt using a 50 μl syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours. A reflux condenser was then inserted between the gas joint and the flask in the prepolymer apparatus described above. The molten prepolymer was dissolved by adding 100 mL of chloroform to the reaction flask with stirring.

Next, 6.9 mL of triethylamine (TEA) and 1.21 g of DMAP were added to the stirring reaction mixture. The reaction mixture was then chilled to about 4° C. in an ice bath. A solution of approximately 2.5 mL of freshly distilled ethyl dichlorophosphate (EOPCl₂) in 25 mL of chloroform was prepared in a dropping funnel. The solution in the funnel was added drop wise to the reaction mixture over a period of about 30 minutes. After the addition was complete the reaction mixture was allowed to continue stirring at about 4° C. for 10 minutes and then the ice bath was removed. The reaction mixture was allowed to warm to room temperature over about 1 hour. At this time a significant increase in viscosity of the clear solution was observed. The reaction mixture was then heated to reflux using an oil bath. Over the next hour the solution became cloudy. The reaction mixture was allowed to reflux over two nights, about 38 hours total.

At this time, a Barret trap was inserted between the condenser and the flask and 88 mL of solvent (⅔ of the total volume) were distilled from the reaction mixture. The Barret trap was removed and the reaction mixture was allowed to reflux for an additional 16 hours with the oil bath temperature between 98-102° C. Next, the oil bath temperature was increased to 115° C. for 2 hours. After this time, the reaction mixture was allowed to cool to room temperature, and 200 mL of dichloromethane was added and transferred to a separatory funnel. The reaction mixture was extracted twice with 100 mL of 0.1 M HCl and twice with 100 mL of saturated sodium chloride solution. The organic layer was isolated, dried overnight in the freezer at about −15° C. over 50 g of sodium sulfate, and filtered twice. The resulting polymer solution was poured into 1500 mL of hexane plus 500 mL of ether. The resulting mass of polymer was dried under vacuum. The Inherent Viscosity (IV) of this material was measured to be 0.39 dL/g.

### EXAMPLE 2

### Second Synthesis of D,L-PL(PG)EOP

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round-bottom flask. The flask was equipped with a gas joint and a stirrer bearing/shaft/paddle assembly. The mixture was evacuated and filled with argon five times to remove residual air and moisture. Each time the polymerization vessel was evacuated to a pressure between 0.5 and 10 Torr. The reaction apparatus was immersed in a preheated oil bath at 125° C., connected to an argon source with an oil bubbler, and stirred at a moderate speed until all of the solid monomer had melted. At this time, a volume of stock stannous octoate solution (about 130 mg/ml in toluene) equivalent to 100 ppm stannous octoate (29 ppm Sn) was added to the melt using a syringe. The reaction mixture was allowed to stir under a slight argon pressure for 3 hours. The oil bath temperature was then reduced to about 105° C. and the residual monomer was removed under vacuum. The pressure was maintained as low as possible, typically between 0.5 and 10 Torr. The upper parts of the reaction assembly were heated gently with a heat gun to aid in the monomer removal. The total time under vacuum was 1 hour.

The prepolymer was cooled to room temperature under argon gas and allowed to stand for 12-18 hours at ambient temperature. The prepolymer was dissolved in 84 ml of chloroform with stirring and 2.5 equivalents of triethylamine (TEA) and 0.5 equivalents of DMAP were added to the stirring reaction mixture using a powder funnel. The reaction mixture was chilled to about −5 to about −15° C. in a cold bath. A solution of about 1 equivalent of distilled ethyl dichlorophosphate (EOPCl₂) in 10 ml of chloroform was prepared in a dropping funnel. The solution in the funnel was added slowly to the reaction mixture over a period of 0.5 hour.

After the addition was complete, the reaction mixture was allowed to stir at low temperature for 1 hour at −5° C. The reaction was then quenched with 1 ml of anhydrous methanol and stirred for another five minutes. Next, the reaction mixture was transferred to a 0.5 gallon vessel and mixed with 37 g of Dowex DR-2030 IER and 30 g of Dowex M-43, and shaken on a mechanical shaker for 2 hour to remove residual DMAP and TEA free base and salts (the IERs had been washed with several bed volumes of methanol and chloroform and dried under vacuum at ambient temperature for about 18 hours). The resin was removed from the reaction mixture by vacuum filtration through Whatman 54 filter paper.

The resin was washed with about one bed volume of dichloromethane and the filtrate was concentrated to approximately 50 ml. The viscous filtrate was poured into 200 ml of petroleum ether to precipitate the polymer. The polymer mass was washed with 100 ml of petroleum ether and dried under vacuum. Molecular weights of the polymers were obtained from gel permeation chromatography (GPC) using both differential refractive index detection and a polystyrene calibration curve (CC) and by light scattering detection. The molecular weight and IV data for the polymers prepared by this process are listed in the table below.

| Sample | Mw (LS), daltons | Mw (CC), daltons | IV, dL/g |
|---|---|---|---|
| 1 | 101,200 | 107,500 | 0.62 |
| 2 | 150,100 | 155,900 | 0.80 |
| 3 | 85,200 | 84,300 | — |
| 4 | 92,600 | 89,900 | — |

### EXAMPLE 3

### Synthesis of D,L-PL(EG)EOP

All glassware was dried for a minimum of 2 hours at 105° C. and allowed to cool in a desiccator or cooled under a stream of argon gas. A 100.0 g portion of D,L-lactide and 4.3 g of ethylene glycol (EG) (molar ratio, 10:1) were weighed into a 1000 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene) equivalent to 120 ppm stannous octoate or 35 ppm Sn was added to the melt using a syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours.

The molten prepolymer was dissolved in 350 ml of chloroform with stirring and 2.5 equivalents of TEA and 0.5 equivalents of DMAP were added to the stirring reaction mixture using a powder funnel. The reaction mixture was chilled to about −5° C. in a cold bath. A solution of about 1 equivalent of distilled ethyl dichlorophosphate (EOPCl₂) in 97 ml of chloroform was prepared in a dropping funnel. The solution in the funnel was added slowly to the reaction mixture over a period of 2 hours. After the addition was complete, the reaction mixture was allowed to stir at low temperature for 45 minutes at −5° C. After 2 hours a significant increase in viscosity of the clear solution was observed. The reaction was then quenched with 6.8 ml of anhydrous methanol and stirred for another five minutes.

Next, the reaction mixture was transferred to a 0.5 gallon vessel and mixed with 87 g of Dowex HCR-S IER and 104 g of Dowex-43, and shaken on a mechanical shaker for 1 hour to remove residual DMAP and TEA free base and salts (the IERs had been washed with several bed volumes of methanol and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 150 ml. The viscous filtrate was poured into 2000 ml of hexane to precipitate the polymer. The polymer mass was washed with 2×200 ml of hexane and dried under vacuum. The molecular weights were determined by GPC were 40,400 for Mw (LS) and 42,000 for Mw (CC).

### EXAMPLE 4

### Synthesis of D,L-PL(HD)EOP

All glassware was dried for a minimum of 2 hours at 105° C. and allowed to cool in a desiccator or cooled under a stream of argon gas. A 100.0 g portion of D,L-lactide and 8.2 g of 1,6-hexane diol (HD) (molar ratio, 10:1) were weighed into a 1000 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution equivalent (about 130 mg/ml in toluene) to 120 ppm stannous octoate or 35 ppm Sn was added to the melt using a syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours.

The molten prepolymer was dissolved in 350 ml of chloroform with stirring and 2.5 equivalents of triethylamine (TEA) and 0.5 equivalents of DMAP were added to the stirring reaction mixture using a powder funnel. The reaction mixture was chilled to about −5° C. in a cold bath. A solution of about 1 equivalent of distilled ethyl dichlorophosphate (EOPCl₂) in 97 ml of chloroform was prepared in a dropping funnel. The solution in the funnel was added slowly to the reaction mixture over a period of 2 hours. After the addition was complete, the reaction mixture was allowed to stir at low temperature for 45 minutes at −5° C. After 2 hours, a significant increase in viscosity of the clear solution was observed. The reaction was then quenched with 6.8 ml of anhydrous methanol and stirred for another five minutes.

Next, the reaction mixture was transferred to a 0.5 gallon vessel and mixed with 87 g of Dowex HCR-S IER and 104 g of Dowex-43, and shaken on a mechanical shaker for 1 hour to remove residual DMAP and TEA free base and salts (the IERs had been washed with several bed volumes of methanol and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 150 ml. The viscous filtrate was poured into 2000 ml of hexane to precipitate the polymer. The polymer mass was washed with 2×200 ml of hexane and dried under vacuum. The molecular weights were determined by GPC were 36,700 for Mw (LS) and 34,100 for Mw (CC). The value for IV was 0.33 dL/g.

### EXAMPLE 5

### Polymer of PG, D,L-Lactide, Glycolide, and Ethyl Dichlorophosphate

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round-bottom flask. The flask was equipped with a gas joint and a stirrer bearing/shaft/paddle assembly and a 125 ml dropping funnel containing 4.6 g of glycolide. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene) equivalent to 3.6 mg tin (120 ppm stannous octoate or 35 ppm tin) was added to the melt using a 50 μl syringe. The reaction mixture was allowed to stir under a slight argon pressure for approximately 16 hours. At this time the glycolide was melted using a heat gun and added to the polymer melt in the flask. The melt was stirred for an additional 2 hours. The oil bath temperature was then reduced to about 115° C. and the residual monomer was removed under vacuum. The upper parts of the reaction assembly were heated gently with a heat gun to aid in the monomer removal. The total time under vacuum was 2 hours.

The molten prepolymer was suspended in 84 ml of chloroform with stirring and 2.5 equivalents of TEA and 0.5 equivalents of DMAP were added to the stirring reaction mixture using a powder funnel. The reaction mixture was chilled to about 4° C. in a cold bath. A solution of about 1 equivalent of distilled ethyl dichlorophosphate (EOPCl₂) in 27.5 ml of chloroform was prepared in a dropping funnel. The solution in the funnel was added slowly to the reaction mixture over a period of 1 hour. After the addition was complete, the reaction mixture was allowed to stir at low temperature for another 1.75 hours and then the cold bath was removed. The reaction mixture was allowed to warm to room temperature and stirred for 2 to 18 hours. After 2 hours a significant increase in viscosity of the clear solution was observed. The reaction was then quenched with 1 ml of anhydrous methanol and stirred for another five minutes.

Next, 37 g of dry Dowex HCR-S IER and 30 g of dry Dowex M-43 were added to the reaction mixture and stirring was continued for another hour to remove residual DMAP and TEA free base and salts. The IERs were removed from the reaction mixture by vacuum filtration through Whatman 54 filter paper. The resin was washed with about one bed volume of dichloromethane and the filtrate was concentrated to approximately 50 ml. The viscous filtrate was poured into 700 ml of petroleum ether to precipitate the polymer and dried under vacuum.

### EXAMPLE 6

### Synthesis of D,L-PL(PG)HOP

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene) equivalent to 3.6 mg tin (120 ppm stannous octoate or 35 ppm tin) was added to the melt using a 50 μl syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours.

The molten prepolymer was dissolved in 100 ml of chloroform with stirring and TEA and DMAP were added to the stirring reaction mixture using a powder funnel. The funnel was rinsed with 10 ml of chloroform. The reaction mixture was chilled to about 4° C. in a cold bath. A solution of about 1 equivalent of distilled hexyl dichlorophosphate (HOPCl₂) in 27.5 ml of chloroform was prepared in a dropping funnel. The solution in the funnel was added slowly to the reaction mixture over a period of 1 hour. After the addition was complete, the reaction mixture was allowed to stir at low temperature for another hour and then the cold bath was removed. The reaction mixture was allowed to warm to room temperature and stirred for 2 to 18 hours. After 2 hours a significant increase in viscosity of the clear solution was observed. The reaction was then quenched with 800 11 of anhydrous methanol and stirred for another five minutes.

Next, Dowex MR-3C ion exchange resin (IER) was added to the reaction mixture and stirring was continued for another hour to remove residual DMAP and TEA free base and salts (the Dowex resin had been washed with several bed volumes of methanol and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 100 ml. The viscous filtrate (now a somewhat cloudy solution) was poured into 1000 ml of hexane to precipitate the polymer. The polymer mass was washed with 2×200 ml of hexane and dried under vacuum. The molecular weight and IV data for the polymers prepared by this process are listed in the table below.

| Sample | Mw (LS), daltons | Mw (CC), daltons | IV, dL/g |
|---|---|---|---|
| 1 | 64,200 | 58,000 | 0.48 |
| 2 | 68,000 | 62,700 | 0.43 |

### EXAMPLE 7

### Synthesis of D,L-PL(PG)EP

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 130° C., connected to an argon source with an oil bubbler, and stirred at a moderate speed until all of the solid monomer had melted.

At this time, a volume of stock stannous octoate solution (about 130 mg/ml in toluene) equivalent to 120 ppm stannous octoate or 35 ppm Sn was added to the melt using a syringe. The reaction mixture was allowed to stir under a slight argon pressure for 4 hours. The oil bath temperature was then reduced to about 110° C. and the residual monomer was removed under vacuum. The upper parts of the reaction assembly were heated gently with a heat gun to aid in the monomer removal. The total time under vacuum was 2 hours.

The molten prepolymer was dissolved in 84 ml of chloroform with stirring and 2.5 equivalents of TEA and 0.5 equivalents of DMAP were added to the stirring reaction mixture using a powder funnel. The reaction mixture was chilled to about −5° C. in a cold bath. A solution of about 1 equivalent of distilled ethyl dichlorophosphonate (EPCl₂) in 9 ml of chloroform was prepared in a dropping funnel. The solution in the ftmnel was added slowly to the reaction mixture over a period of 0.5 hour. After the addition was complete, the viscosity of the solution had increased significantly and the reaction mixture was allowed to stir at low temperature for 1 hour at −5° C. The reaction was then quenched with 1 ml of anhydrous methanol and stirred for another five minutes.

Next, the reaction mixture was transferred to a 0.5 gallon vessel and mixed with 37 g of Dowex DR-2030 IER and 30 g of Dowex-43, and shaken on a mechanical shaker for 2 hour to remove residual DMAP and TEA free base and salts (the IERs had been washed with several bed volumes of methanol and chloroform and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 50 ml. The viscous filtrate was poured into 200 ml of petroleum ether to precipitate the polymer. The polymer mass was washed with 100 ml of petroleum ether and dried under vacuum. The molecular weight data for the polymers prepared by this process are listed in the table below.

| Sample | Mw (LS), daltons | Mw (CC), Daltons |
|---|---|---|
| 1 | 339,900 | 327,600 |
| 2 | 369,800 | 360,900 |

### EXAMPLE 8

### Synthesis of P(cis- and trans-CHDM/HOP)

All glassware was dried for a minimum of two hours at 105° C. and allowed to cool in a desiccator or cooled under a stream of argon gas. A reaction assembly consisting of a 1 L three-neck round-bottom flask equipped with a gas joint, a stirrer bearing/shaft/paddle and a dropping funnel. A solution of 20.0 g of 1,4-cyclohexane dimethanol (CHDM) was prepared in 75 ml of anhydrous tetrahydrofuran (THF) and transferred to the reaction vessel. The beaker was rinsed with 25 ml of THF and the wash was transferred to the reaction vessel.

Next, 29.0 ml of N-methylmorpholine (NMM) and 1.61 g of DMAP were added to the reaction mixture through a powder funnel. A solution of 28.86 g of hexyl dichlorophosphate (HOPCl₂) in 30 ml of THF was prepared under argon and transferred to the dropping funnel while the reaction mixture was cooled to 4° C. in a cold bath. The solution in the funnel was added to the reaction mixture over a period of one hour. With 5 to 10 minutes after the start of addition, a white precipitate, presumably the hydrochloride salts of NMM and DMAP, began to form. After the addition was complete the funnel was rinsed with 30 ml of THF. The reaction mixture was stirred for 1 hour at 4° C. and then for either 2 or 18 hours at ambient temperature.

At the prescribed time, the precipitate was removed from reaction mixture by vacuum filtration. The filtrate was diluted with 100 ml of dichloromethane, transferred to a half-gallon jar and 86.5 of dried Dowex HCR-S IER and 103.8 g of dried Dowex M-43 IER were added to the filtrate. The jar was sealed with a Teflon lined lid and the mixture was agitated on a mechanical shaker for two hours.

At this time, the IERs were removed by vacuum filtration and the filtrate was concentrated to approximately 100 ml under vacuum. The polymer solution was poured in 2 L of hexame and the resulting fluid material that precipitated was isolated and transferred to a Teflon lined glass dish. The polymer was dried under vacuum to yield a sticky, free flowing viscous liquid. The Mw (LS) data for the polymers prepared by this process are listed in the table below.

| Sample | Mw (LS), daltons | Mw (CC), daltons | IV, dL/g |
|---|---|---|---|
| 1 | 4400 | 5500 | 0.14 |
| 2 | 5000 | 6500 | 0.11 |
| 3 | 4000 | 4600 | 0.10 |

### EXAMPLE 9

### Synthesis of P(BHET/EOP)

All glassware was dried for a minimum of two hours at 105° C. and allowed to cool in a desiccator cooled under a stream of argon gas. A reaction assembly consisting of a 500 ml three-neck round-bottom flask equipped with a gas joint, a stirrer bearing/shaft/paddle and a dropping funnel. First, 30.0 g of bis(hydroxyethyl) terephthalate (BHET) and 28.83 g of DMAP were added to the reaction vessel using a powder funnel and mixed with 81 ml of THF. The solids were dissolved with stirring and gentle heating using a heat gun.

After all solids had dissolved, the reaction mixture was cooled to 4° C. in a cold bath. A solution of 19.2 g of ethyl dichlorophosphate (EOPCl₂) in 24 ml of THF was prepared in a 125 ml addition funnel. The solution in the funnel was added to the solution in the flask over a period of 1 hour. Shortly after the addition had begun, a white precipitate, presumably DMAP hydrochloride, began to precipitate from the reaction mixture. After all of the solution in the funnel had been added, the stirrer shaft/paddle became entrapped in a thick, stiff precipitate and stirring ceased. It appears the polymer that had formed at this time was insoluble in the reaction mixture.

Next, 125 ml of dichloromethane were added and the reaction mixture was swirled by hand until mechanical stirring could be resumed. The reaction mixture was now a homogenous solution containing a white free flowing powder. The reaction mixture was stirred at 4° C. for one hour. The cold bath was removed and the reaction mixture was allowed to warm to ambient temperature and stirred for 16 hours. At this time, the white precipitate was removed from the reaction mixture by vacuum filtration and the filter cake was washed with 100 ml of dichloromethane.

The resulting filtrate was transferred to a half-gallon jar and treated with 156.92 g of undried Dowex HCR-S IER and 160.92 g of undried Dowex M-43 IER. The resins were washed with 2 bed volumes of methanol and 2 bed volumes of dichloromethane prior to use. The jar was sealed with a Teflon lined lid and shaken on a mechanical shaker for two hours. The resin was removed by vacuum filtration and the filtrate, ˜600 ml, was concentrated to ˜150 ml. The clear solution was poured into 1.2 L of hexane. The thick oil that precipitated was washed with 400 ml of hexane and transferred to a Teflon lined glass dish, dried under vacuum. The molecular weights were determined by GPC were 2200 for Mw (LS) and 2100 for Mw (CC). The value obtained for IV was 0.10 dL/g.

### EXAMPLE 10

### Synthesis of P(BHET-EOP/TC)

All glassware was dried for a minimum of two hours at 105° C. and allowed to cool in a desiccator or cooled under a stream of argon gas. A reaction assembly consisting of a 500 ml three-neck round-bottom flask equipped with a gas joint, a stirrer bearing/shaft/paddle and a dropping funnel. First, 30.0 g of BHET and 28.83 g of DMAP were added to the reaction vessel using a powder funnel and mixed with 81 ml of THF and 125 ml of dichloromethane.

The solids were dissolved with stirring and gentle heating using a heat gun. After all solids had dissolved, the reaction mixture was cooled to 4° C. in a cold bath. A solution of 19.2 g of EOPCl₂ in 24 ml of THF was prepared in a 125 ml addition funnel. The solution in the funnel was added to the solution in the flask over a period of 1 hour. Shortly after the addition had begun, a white precipitate, presumably DMAP hydrochloride, began to precipitate from the reaction mixture. The reaction mixture was stirred at 4° C. for one hour. Next, a solution of 4.79 g of terephthaloyl chloride (TC) in 18 ml of THF was prepared in the addition funnel and added to the solution in the flask over a 30-minute period. The reaction mixture was stirred for one hour at 4 □C.

At this time the cold bath was removed and the reaction was allowed to warm to room temperature and stir for another 20 hours. At this time, the white precipitate was removed from the reaction mixture by vacuum filtration. The resulting filtrate was transferred to a half-gallon jar and treated with 88.5 g of dried Dowex HCR-S IER and 73.8 g of dried Dowex M-43 IER. The jar was sealed with a Teflon-lined lid and shaken on a mechanical shaker for two hours. The resin was removed by vacuum filtration and the filtrate was concentrated to ˜100 ml. The clear solution was poured into 2 L of hexane. The thick oil that precipitated was transferred to a Teflon-lined glass dish, dried under vacuum. The molecular weights were determined by GPC were 7200 for Mw (LS) and 4000 for Mw (CC). The value obtained for IV was 0.09 dL/g.

### EXAMPLE 11

### Large-Scale Preparation of D,L-PL(PG)EOP

A 100 g portion of propylene glycol was added to a 3000 ml 3-necked round bottom flask equipped with a gas joint, a stirrer bearing/shaft/paddle assembly, and a Teflon-coated thermocouple. The reaction apparatus was placed in a preheated oil bath at 130° C. and purged with nitrogen for one minute. A 2000 g portion of D,L-lactide was added using a powder addition funnel over a period of 45 minutes. The reaction apparatus was then immersed in the oil so that the oil level was at the bottom of the ground glass joints. The mixture was stirred until all of the solid monomer had melted and the internal temperature had reached approximately 125° C. At this time, a volume of solution of stannous octoate in chloroform equivalent to approximately 400 ppm (117 ppm Sn) was added to the melt using a syringe. The mixture was allowed to stir for approximately 3-16 hours. Then oil bath set point was decreased to approximately 125° C. and any residual unreacted monomer removed using vacuum over approximately 1 hour.

A 2500 ml portion of chloroform was used to dissolve and transfer the prepolymer to a pre-chilled, 20-liter jacketed reactor, which contained 2.5 equivalents (based on propylene glycol) of triethylamine and 0.5 equivalents of DMAP dissolved in 3600 ml of chloroform. The reactor was equipped with a stirrer bearing/shaft/turbine assembly, a gas joint, a tubing adapter, and a Teflon-coated thermocouple. With stirring and chilled recirculation on the jacket, the solution was cooled to below −15° C. A solution of 1 equivalent (based on propylene glycol, approximately 215 g) of distilled ethyl dichlorophosphate (EOPCl₂) in 650 ml chloroform was prepared in a 1000 ml 3-necked round bottom flask equipped with a tubing adapter and a gas joint. The EOPCl₂/chloroform solution was added using a piston pump and Teflon tubing over a period of 50 minutes, maintaining the internal temperature at approximately −10° C. Tubing was connected to the gas joints of the flask and reactor to equalize the pressure during the addition. Following the addition, a 50 ml portion of chloroform was added to rinse the flask, feed lines, and pump. The reaction mixture was stirred for 1 hour at low temperature (−8° C. after 1 hour) before the reaction was quenched with 140 ml of anhydrous methanol.

The reactor was then charged with 3 kg of Dowex DR-2030 IER and 3 kg of Dowex M-43 wetted with approximately 6.5 liters of methylene chloride. The polymer/resin mixture was mixed at low temperature for 3-15 hours, after which it was transferred by vacuum to a stainless steel laboratory Nutsche filter. After filtering off the resin, the polymer solution was pulled through the in-line 8 micron cartridge filter into the concentrator (a similar 10-liter jacketed reactor) where the solution was concentrated with the aid of heated recirculating fluid on the jacket. The 20-liter reactor and the resin in Nutsche were washed with 5 liters of methylene chloride, which were transferred to the concentrator after being stirred for 1 hour. An additional 5 liters of methylene chloride were added to the resin in the Nutsche and added to the concentrator when the solution had been reduced to approximately 6 liters.

Concentration of the polymer solution continued until approximately 4-5 liters of a viscous solution remained. A portion of 1500 ml of ethyl acetate was then added to the polymer solution. The mixture was mixed until homogenous and precipitated in approximately 10 liters of petroleum ether. After the precipitation mixture was stirred for approximately 5 minutes, the supernatant liquid was decanted. The polymer was then washed with 5 liters of petroleum ether. After the mixture was stirred for 5 minutes. The liquid was again decanted. The polymer was poured into a Teflon-coated pan and placed in the vacuum oven at NMT 50° C. After drying for 24 hours, the polymer was ground into smaller pieces and dried for additional time in a vacuum oven at ambient temperature.

### EXAMPLE 12

### Encapsulating Paclitaxel into the Subject Polymers

The term “PACLIMER” shall refer to a subject polymer in a microsphere form with the D,L-PL(PG)EOP composition containing paclitaxel at certain loading levels. The D,L-PL(PG)EOP polymer in PACLIMER may be prepared using the method described in Example 1, 2 or 11, with Examples 2 and 11 being the preferred method of synthesis. The loading level of paclitaxel will be expressly stated or alternatively indicated in parentheses as shown for the following examples: for 30% loading level, “PACLIMER (30%)”; for fifty percent loading, “PACLIMER (50%)”; etc. If there is no loading level indicated for any reference to PACLIMER, then a 10% loading level was used. All microspheres of PACLIMER, unless otherwise indicated, were prepared using the solvent dilution method described below.

The four methods listed below may be applied to a variety of drug for a range of loading levels:

Method 1—Spray Drying:

10 g of a phosphorous linked polymer, e.g., D,L-PL(PG)EOP, is dissolved in methylene chloride at a concentration of about 10%. After the polymer is completely dissolved, an appropriate amount of paclitaxel powder (e.g., 1.1 g for 10% loading, 4.2 g for 30% loading, 10 g for 50% loading, etc.) is added to the solution and stirred until the powder is completely dissolved. Microspheres are then prepared using a spray-drying technique, e.g., using a Buchi Mini Spray Dryer (Model B-191) at inlet temperature of 35° C., pump rate of 16%(˜10 gm/min) for polymer solution and 800 L/hr for atomizer gas (nitrogen), and aspiration at 50% (˜20 mbar). In most instances, the mean diameter of the resulting microspheres for PACLIMER at various loading levels is less than about 20 microns.

Method II—Solvent Evaporation:

Microparticles of the subject compositions will be prepared by solvent evaporation. For example, the subject polymer composition and paclitaxel are dissolved in ethyl acetate, the ethyl acetate solution is then emulsified into a 0.5% polyvinylalcohol (PVA) solution presaturated with ethyl acetate at a stirring rate of 600 rpm, followed by the application of a vacuum (e.g., about 15 inches of Hg) to remove the ethyl acetate. For one preferred process, the ethyl acetate concentration will be reduced to below 10% within 10 minutes. Microparticles will be washed on an appropriate sieve with deionized water and thereafter lyophilized.

Method III—Solvent Dilution:

Microspheres may be prepared by a solvent dilution method using an in-line homogenizer. For example, approximately 50 grams of paclitaxel and 450 grams of subject polymer composition were weighed and dissolved in IL of ethyl acetate. The non-solvent phase was pre-saturated with ethyl acetate; ethyl acetate (800 ml) was added to 9 liter of 0.5% PVA and homogenized for 1 minute. The paclitaxel-subject polymer composition solution and the PVA-ethyl acetate solution were pumped simultaneously through an in-line homogenizer into a container at rates of 1 and 3 liters/min, respectively. The combined solution was gently stirred with an overhead stirrer. Approximately 90 liters of water was added to the container at a rate of 3 L/min. The solution was then gently stirred for 30 minutes. The microsphere suspension was transferred to a filtering/drying unit containing 150 μm scalping and 25 μm product sieves. The resulting microspheres were rinsed with 5 liters of de-ionized water and dried for 3 days under vibration, vacuum and a nitrogen purge. The dried microspheres on the 25 μm sieve were collected into a container.

Method IV—Freeze/Pulverize:

Microparticles are prepared by evaporating the drug/polymer in solution at 40° C. under a nitrogen purge to obtain viscous mass which is subsequently cooled to −40° C., lyophilized, e.g., for 48 hours, and pulverized to a desired size for the microparticles.

### EXAMPLE 13

### Evaluation of Efficacy and Ascites Reduction by PACLIMER in an HT29 Colon Cancer Xenograft

Drugs Tested.

PACLIMER was provided as in powder and suspended in the vehicle (0.1% Tween80 in normal saline) just before administration. Paclitaxel was purchased from Bristol-Myers Squib Co., which was provided as a solution at a concentration of 6 mg/ml. The paclitaxel solution was diluted to 1 mg/ml with 0.9% sodium chloride just before use.

Cell Line and Colon Cancer Mouse Model.

The human colon carcinoma cell line HT29 was used to set up the colon cancer xenograft mouse model. SCID mice were injected i.p. with 10 million cells. This generally results in death from the tumor metastasis and ascites around 30 days after tumor implantation. HT29 cells growing as a monolayer were harvested with trypsin and washed with PBS. The packed cells were resuspended in PBS (10 million cells per ml PBS). 48 SCID mice (C.B.17/Icr SCID, 8-10 weeks of age) were injected i.p. with 1 ml of the HT29 cell suspension (10 million cells for each mouse). 4 days after tumor implantation, the animals were grouped randomly. The 5 groups included: (1) Ascites control: 12 mice (no treatment); (2) Ascites treated: 12 mice (PACLIMER, 800 mg/kg; i.e., active ingredient paclitaxel 80 mg/kg); (3) Survival control: 8 mice (no treatment); (4) Survival PACLIMER: 8 mice (PACLIMER, 800 mg/kg); and (5) Survival paclitaxel: 8 mice (paclitaxel, 40 mg/kg).

Drug Administration and Observation.

For the ascites study, PACLIMER was to be given when obvious ascites was found in the mice. In this study, the ascites was obvious in almost all the mice on day 18.Hence 12 mice were injected with PACLIMER solution and another 12 mice were used as ascites controls. The mice were checked for girth and weight every 5 days after drug administration. On 1, 5, and 10 days after treatment, 4 mice in each group was sacrificed at each time point and the ascites volumes were measured. For the survival study, PACLIMER and paclitaxel were injected IP on day 4 post tumor cell implantation. Then the mice were observed for survival.

Results

The ascites volume of PACLIMER treated group versus control group is shown in Table 1.

TABLE 1
Ascites Volume of Mice, PACLIMER vs. Control (ml)

| | Control | | | | PACLIMER | | | |
|---|---|---|---|---|---|---|---|---|
| Day 19 | 3.5 | 3.5 | 3.5 | 2.5 | 3 | 2 | 5 | 2.5 |
| Day 23 | 1 | 1.2 | 1 | 1 | 0.7 | 0.7 | 1.4 | 0.7 |
| Day 27 | 1 | 0.8 | 0.7 | | 0 | 0 | 0.9 | 0 |

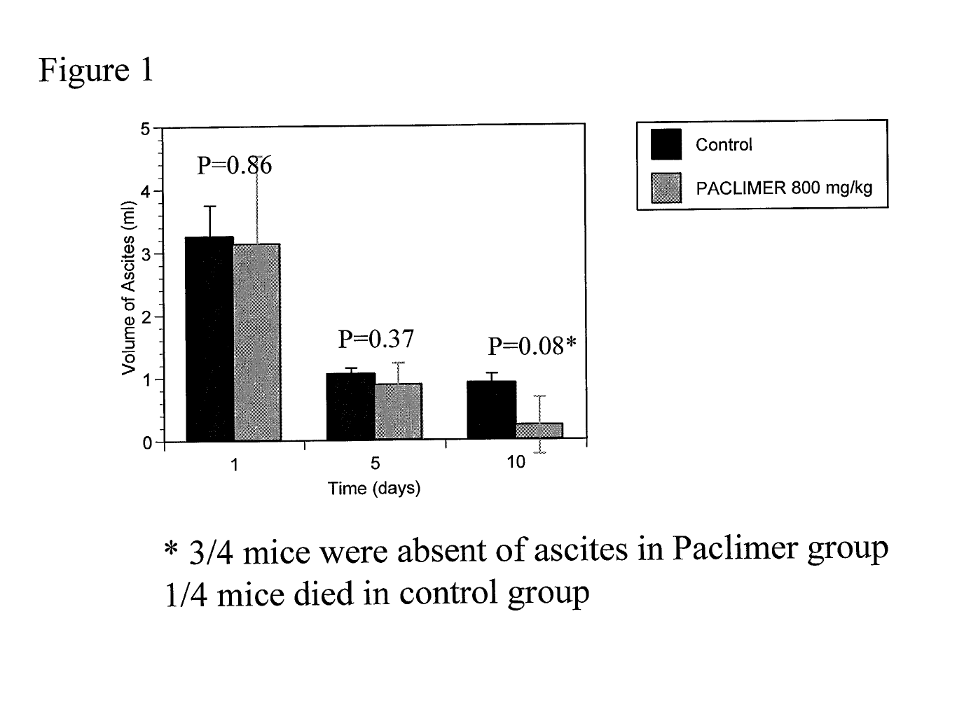

These data are illustrated in FIG. 1. It appears that PACLIMER could reduce the ascites volume in the HT29 mouse model. 3 out of 4 mice in the treated group had no ascites 10 days after PACLIMER treatment. It is noted that animals with the most apparent ascites were euthanised at the initial time point, i.e. day 19.

The girth and body weights of the mice are presented in Table 2 and Table 3 respectively.

TABLE 2
The girth measurement of mice, PACLIMER vs. Control (mm)

| Control | | | | | | | | | | | | | Control |
|---|---|---|---|---|---|---|---|---|---|---|---|---|---|
| (in mm) | | | | | | | | | | | | | Average |
| day 13 | 18 × 16 | 21 × 17 | 17 × 14 | 19 × 16 | 20 × 17 | 20 × 16 | 19 × 16 | 18 × 16 | 20 × 17 | 18 × 15 | 20 × 17 | 19 × 15 | 19.1 × 16.0 |
| day 19 | 20 × 15 | 22 × 15 | 19 × 14 | 21 × 15 | 22 × 16 | 21 × 15 | 20 × 16 | 22 × 16 | 20 × 17 | 20 × 16 | 20 × 18 | | 20.6 × 15.7 |
| day 23 | 21 × 16 | 20 × 16 | 19 × 14 | 21 × 16 | 23 × 20 | 20 × 15 | 20 × 16 | | | | | | 20.6 × 16.1 |
| day 27 | 21 × 16 | 23 × 15 | 19 × 14 | | | | | | | | | | 21.0 × 15.0 |
| PACLIMER | | | | | | | | | | | | | PACLIMER |
| (in mm) | | | | | | | | | | | | | Average |
| day 13 | 18 × 15 | 16 × 15 | 19 × 15 | 16 × 15 | 19 × 16 | 19 × 14 | 19 × 16 | 15 × 15 | 20 × 16 | 20 × 16 | 22 × 16 | 19 × 16 | 18.5 × 15.4 |
| day 19 | 17 × 16 | 19 × 15 | 18 × 15 | 19 × 15 | 21 × 16 | 20 × 15 | 20 × 15 | 21 × 16 | 19 × 17 | 22 × 18 | 22 × 20 | 20 × 15 | 19.9 × 16.1 |
| day 23 | 18 × 13 | 19 × 13 | 19 × 14 | 18 × 14 | 20 × 15 | 19 × 13 | 18 × 13 | 21 × 15 | | | | | 19.0 × 13.7 |
| day 27 | 19 × 14 | 18 × 14 | 19 × 12 | 18 × 14 | | | | | | | | | 18.5 × 13.5 |

TABLE 3
Weight of mice (gram)

| | | | | | | | | | | | | | Control |
|---|---|---|---|---|---|---|---|---|---|---|---|---|---|
| Control Weight (gm) | | | | | | | | | | | | | Average |
| day 13 | 21.2 | 22.4 | 15.1 | 22 | 22.6 | 22.4 | 21.1 | 22.2 | 23.4 | 19.5 | 23.1 | 20.5 | 21.3 |
| day 19 | 20.6 | 19.9 | 15 | 20.4 | 20.8 | 22.3 | 20.8 | 20.2 | 23.7 | 19.8 | 22.5 | | 20.5 |
| day 23 | 18.3 | 19.1 | 13.8 | 20.3 | 21.2 | 19.5 | 18.1 | | | | | | 18.6 |
| day 27 | 18.1 | 17.9 | 13.4 | | | | | | | | | | 16.5 |
| | | | | | | | | | | | | | PACLIMER |
| PACLIMER Weight (gm) | | | | | | | | | | | | | Average |
| day 13 | 21.4 | 19.8 | 17.9 | 21.2 | 20.8 | 19.8 | 20.4 | 21.8 | 20.5 | 23.5 | 23.1 | 21.9 | 21.0 |
| day 19 | 22 | 19.7 | 17.5 | 19.6 | 19.6 | 18.2 | 18.5 | 21.7 | 19.5 | 22 | 22.3 | 22.4 | 20.3 |
| day 23 | 21.3 | 19.8 | 17 | 19.5 | 17.5 | 15.9 | 16.4 | 20.9 | | | | | 18.5 |
| day 27 | 21.2 | 19.8 | 15.3 | 19.6 | | | | | | | | | 19.0 |

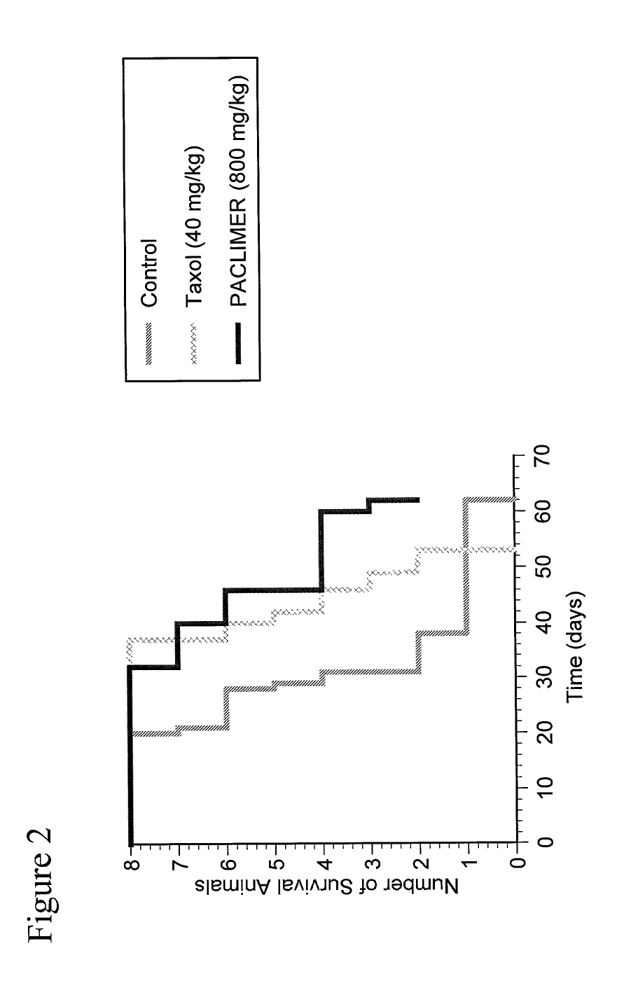

Apparent PACLIMER efficacy based on the survival is shown in Table 4 and FIG. 2.

TABLE 4
SURVIVAL OF MICE AFTER TREATMENT WITH
PACLIMER AND PACLITAXEL IN THE HT29 MOUSE MODEL

| Treatment Group | Control | PACLIMER 80 mg/kg | Paclitaxel 40 mg/kg |
|---|---|---|---|
| Median Survival in Days | 30 | 53 | 44 |

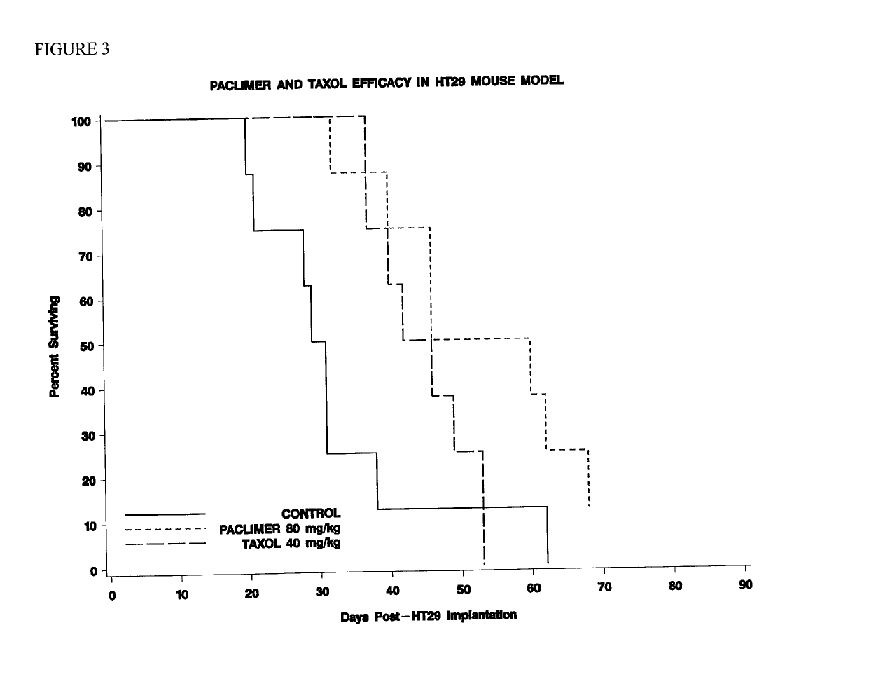

The Kaplan-Meier survival curves were calculated and are displayed in FIGS. 2 and 3, based on the data shown in Table 4. PACLIMER drug treatment was statistically different from control (p=0.0101). The comparison between paclitaxel and control did not reach statistical significance (p=0.1308). In addition, pairwise comparisons between drug treatments revealed that PACLIMER treatment was better than paclitaxel treatment in terms of longer survival time (p=0.0855).

### 7. EQUIVALENTS

Those skilled in the art will recognize, or will be able to ascertain using no more than routine experimentation, a number of equivalents to the specific embodiments and practices of the invention described herein. Such equivalents fall within the scope of the present invention and are encompassed by the following claims.

1. A method for treating a malignant effusion within a body cavity of a patient, comprising:
instilling into said body cavity a therapeutically effective amount of a composition comprising a biocompatible polymer and an antineoplastic taxane.

2. The method of claim 1, wherein said polymer is biodegradable.

3. The method of claim 1, wherein said polymer comprises a polymer having phosphorous-based linkages.

4. The method of claim 3, further comprising positioning said composition in at least partial contact with at least a portion of said malignant effusion or tissue surrounding said malignant effusion.

5. The method of claim 3, wherein the median survival rate of said patient from said malignant effusion is increased by at least about 10% as compared with the median survival rate obtained by administration of a composition comprising about the same effective dosage of said antineoplastic taxane without said polymer.

6. The method of claim 4, wherein the median survival rate of said patient from said malignant effusion is increased by at least about 20% as compared with the median survival rate obtained by administration of a composition comprising said antineoplastic taxane without said polymer.

7. The method of claim 1, wherein the median survival rate of said patient from said malignant effusion is increased by at least about 30% as compared with the median survival rate obtained by administration of a composition comprising the same dosage of said antineoplastic taxane without said polymer.

8. The method of claim 1, wherein said body cavity is one of the following: the intrapleural space, the pericardial space, or the peritoneal cavity.

9. The method of claim 1, wherein an access device is used to instill said composition into said body cavity.

10. The method of claim 3, further comprising draining said malignant effusion from said body cavity.

11. The method of claim 1, wherein said composition contains at least about 10 percent by weight of the sum of the weight of said antineoplastic taxane and said biocompatible polymer.

12. The method of claim 3, wherein said composition contains at least about 5 percent to about 60 percent by weight of the sum of the weight of said antineoplastic taxane and said biocompatible polymer.

13. The method of claim 1, wherein said composition is in the form of microparticles.

14. The method of claim 3, wherein said composition is formulated as microspheres.

15. The method of claim 14, wherein the mean diameter of said microspheres is less than about 250 microns.

16. The composition of claim 14, wherein the mean diameter of said microspheres is less than about 100 microns.

17. The method of claim 14, wherein said microspheres are mixed with a pharmaceutically acceptable carrier before said instillation.

18. The method of claim 3, wherein said composition provides extended release of said antineoplastic taxane.

19. The method of claim 3, wherein a single dose of said composition provides extended release of a therapeutically effective amount of said antineoplastic taxane for a period of at least about 15 days after instillation.

20. The method of claim 1, wherein a single dose of said composition provides extended release of said antineoplastic taxane over a period of at least about 30 days.

21. The method of claim 20, wherein said antineoplastic taxane comprises at least about 10% by weight of the sum of the weight of said antineoplastic taxane and said biocompatible polymer.

22. The method of claim 19, wherein said antineoplastic taxane comprises at least about 5% by weight of the sum of the weight of said antineoplastic taxane and said biocompatible polymer.

23. The method of claim 18, wherein said composition is in at least partial contact with at least a portion of said malignant effusion or tissue surrounding said malignant effusion.

24. The method of claim 1, wherein said polymer has five or more units represented by the following formula: 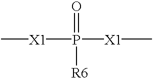
wherein, independently for each occurrence of said monomeric unit:
X1, each independently, represents —O— or —N(R5)—;
R5 represents —H, aryl, alkenyl or alkyl; and
R6 is any non-interfering substituent.

25. The method of claim 24, wherein each occurrence of X1 for each of said units represents O.

26. The method of claim 25, wherein each occurrence of R6 for each of said units represents H, alkyl, —O-alkyl, —O-cycloalkyl, aryl, —O-aryl, heterocycle or —O-heterocycle.

27. The method of claim 1, wherein said polymer has two or more monomeric units represented by the following Formula V: 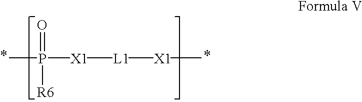
wherein, independently for each occurrence of said monomeric unit:
X1, each independently, represents —O— or —N(R7)—;
R7 represents —H, aryl, alkenyl or alkyl;
L1 represents any chemical moiety that does not materially interfere with the biocompatibility of said polymer;
R8 represents —H, -alkyl, —O-alkyl, -cycloalkyl, —O-cycloalkyl, -alkenyl, —O-alkenyl, -cycloalkenyl, —O-cycloalkenyl, -aryl, —O-aryl, -heterocycle, —O-heterocycle, -polycycle, —O-polycycle, or —N(R9)R10;
R9 and R10, each independently, represent a hydrogen, an alkyl, an alkenyl, —(CH₂)ₘ—R11, or R9 and R10, taken together with the N atom to which they are attached complete a heterocycle having from 4 to about 8 atoms in the ring structure;
m represents an integer in the range of 0-10; and
R11 represents —H, -alkyl, -alkenyl, -aryl, -cycloalkyl, -cycloalkenyl, -heterocycle or -polycycle.

28. The method of claim 27, wherein said polymer comprises at least about five of said monomeric units.

29. The method of claim 28, wherein all X1 are O.

30. The method of claim 29, wherein L1 for at least a plurality of said units has 2 to about 20 atoms of carbon, oxygen, sulfur and nitrogen, wherein at least 60 percent of said atoms are carbon.

31. The method of claim 1, wherein said polymer has one or more monomeric units represented by the following Formula VI: 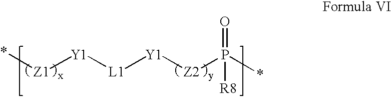
wherein Z1 and Z2, respectively, for each independent occurrence is: 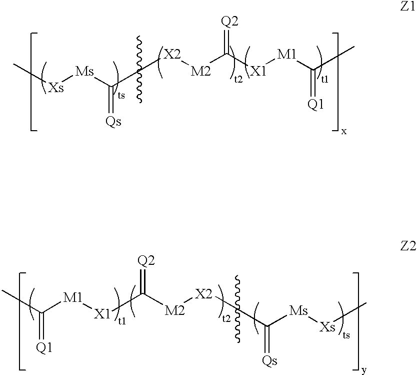
wherein, independently for each occurrence of said monomeric unit:
Q1, Q2 . . . Qs, each independently, represent —O— or —N(R7);
X1, X2 . . . Xs, each independently, represent —O— or —N(R7);
R7 represents —H, aryl, alkenyl or alkyl;
the sum of t1, t2 . . . ts is an integer and equal to at least one or more;
Y1 represents —O—, —S— or —N(R7)—;
x and y are each independently integers from 1 to about 1000 or more;
L1 represents any chemical moiety that does not materially interfere with the biocompatibility of said polymer;
M1, M2 . . . Ms each independently, represents any chemical moiety that does not materially interfere with the biocompatibility of said polymer;
R8 represents —H, -alkyl, —O-alkyl, -cycloalkyl, —O-cycloalkyl, -alkenyl, —O-alkenyl, -cycloalkenyl, —O-cycloalkenyl, -aryl, —O-aryl, -heterocycle, —O-heterocycle, -polycycle, —O-polycycle, or —N(R9)R10;
R9 and R10, each independently, represent a hydrogen, an alkyl, an alkenyl, —(CH₂)ₘ-R11, or R9 and R10, taken together with the N atom to which they are attached complete a heterocycle having from 4 to about 8 atoms in the ring structure;
m represents an integer in the range of 0-10; and
R11 represents —H, -alkyl, -alkenyl, -aryl, -cycloalkyl, -cycloalkenyl, -heterocycle or -polycycle.

32. The method of claim 31, wherein said monomeric units comprise at least about 95 percent of the repeating units of said polymer.

33. The method of claim 32, wherein the average molar ratio of (x or y):L1, when ts is equal to one, is from about 10:1 to about 4:1.

34. The method of claim 32, wherein L1 represents a divalent branched or straight chain or cyclic aliphatic group or divalent aryl group.

35. The method of claim 32, wherein each Q1, Q2 . . . Qs and each X1, X2 . . . Xs of each of said monomeric units of said polymer is O and the sum of t1, t2 . . . ts equals one for each of Z1 and Z2.

36. The method of claim 35, wherein each M1, M2 . . . Ms of each of said monomeric units of said polymer represents a divalent aliphatic moiety having from 1 to about 7 carbon atoms.

37. The method of claim 31, wherein said monomeric units are represented by the following Formula VIf:

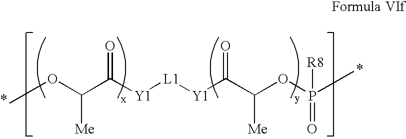

38. The method of claim 31, wherein each of Z1 and Z2 is represented by: 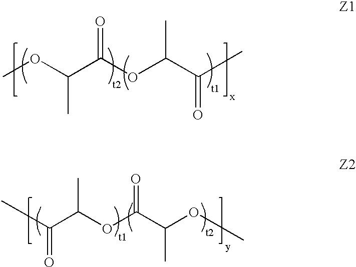
wherein the configuration of the chiral carbons independently for each unit x for Z1 and unit y for Z2 is either D for t1 and L for t2, or L for t1 and D for t2.

39. The method of claim 38, wherein each of Y1 is O and L1 is —CH(CH₃)CH₂—.

40. The method of claim 1, wherein said polymer has one or more monomeric units represented by the following Formula VII: 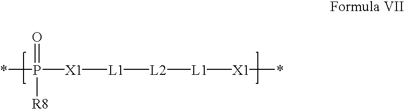
wherein, independently for each occurrence of said monomeric unit:
X1, each independently, represents —O— or —N(R7)—;
R7 represents —H, aryl, alkenyl or alkyl;
L1 represents any chemical moiety that does not materially interfere with the biocompatibility of said polymer;
R8 represents —H, -alkyl, —O-alkyl, -cycloalkyl, —O-cycloalkyl, -alkenyl, —O-alkenyl, -cycloalkenyl, —O-cycloalkenyl, -aryl, —O-aryl, -heterocycle, —O-heterocycle, -polycycle, —O-polycycle, or —N(R9)R10;
R9 and R10, each independently, represent a hydrogen, an alkyl, an alkenyl, —(CH₂)ₘ—R11, or R9 and R10, taken together with the N atom to which they are attached complete a heterocycle having from 4 to about 8 atoms in the ring structure;
m represents an integer in the range of 0-10; and
R11 represents —H, -alkyl, -alkenyl, -aryl, -cycloalkyl, -cycloalkenyl, -heterocycle or -polycycle; and
L2 represents a divalent, branched or straight chain aliphatic group, a divalent cycloaliphatic group, a phenylene group, or a group of the formula:

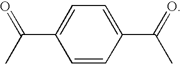

41. The method of claim 40, wherein each of L1 is —CH₂—.

42. The method of claim 41, wherein each X1 of each of said units is O.

43. The method of claim 40, wherein said polymer has one or more monomeric units represented by the following Formula VIII: 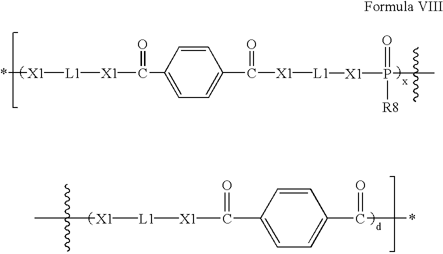
wherein d is equal to one or more and x is equal to or greater than one.

44. The method of claim 43, wherein each L1 independently represents an alkylene group, a cycloaliphatic group, a phenylene group or a divalent group of the formula: 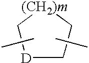
wherein D is O, N or S and m is an integer from 1 to 3.

45. A composition comprising a biocompatible polymer and a therapeutically effective amount of an antineoplastic taxane, wherein said composition is suitable for administration to a patient and wherein said composition is in at least partial contact with at least a portion of a malignant effusion or the tissue surrounding said malignant effusion.

46. The composition of claim 45, wherein said biocompatible polymer is biodegradable.

47. The composition of claim 45, wherein said biocompatible polymer comprises a polymer having phosphorous-based linkages.

48. The use of a composition in the manufacture of a medicament to treat or prevent a malignant effusion in a subject, wherein said composition is one of the compositions claimed above.

49. A kit for treating a malignant effusion in a body cavity, comprising: a therapeutically effective amount of a composition comprising a biocompatible polymer and an antineoplastic taxane; and a drug delivery device dimensionally adapted for instilling said composition into said body cavity.

50. A kit for treating a malignant effusion in a body cavity, comprising: a therapeutically effective amount of a composition comprising a biocompatible polymer and an antineoplastic taxane; and instructions for use of said composition for treating a malignant effusion.
